(19) United States
(12) Patent Application Publication
(10) Pub. No.: US 2010/0179173 A1
(43) Pub. Date: Jul. 15, 2010

(54) SUBSTITUTED FUSED PYRIMIDINES AS ANTAGONISTS OF GPR105 ACTIVITY

(76) Inventors: Daniel Guay, Lachine (CA); Christian Beaulieu, Laval (CA); Michel Belley, Pierrefonds (CA); Sheldon N. Crane, Montreal (CA); Jeancarlo De Luca, Montreal (CA); Rejean Fortin, Montreal (CA); Yves Gareau, Notre-Dane de l'ile-Perrot (CA); Lianhai Li, Pierrefonds (CA); Michel Therien, Laval (CA); Geoffrey K. Tranmer, Westmount (CA); Vouy Linh Truong, Pierrefonds (CA); Zhaoyin Wang, Kirkland (CA)

Correspondence Address: MERCK, P O BOX 2000, RAHWAY, NJ, 07065-0907, US

(21) Appl. No.: 12/663,556

(22) PCT Filed: Jun. 26, 2008
(86) PCT No.: PCT/CA2008/001214

§ 371 Date: Dec. 8, 2009

Related U.S. Application Data
(60) Provisional application No. 60/937,792, filed on Jun. 28, 2007.

(51) Int. Cl. A61K 31/519; C07D 471/04; A61P 3/10

(52) U.S. Cl. 514/264.1; 544/279

(57) ABSTRACT

Fused pyrimidine compounds of structural formula (I) are effective as antagonists of the biological activity of the GPR105 protein. They are useful for the treatment, control or prevention of disorders responsive to antagonism of this receptor, such as diabetes, particularly, Type 2 diabetes, insulin resistance, hyperglycemia, lipid disorders, obesity, atherosclerosis, and Metabolic Syndrome.

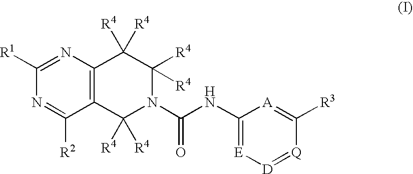

### FIELD OF THE INVENTION

The present invention relates to substituted fused pyrimidine compounds which are antagonists of the biological activity of the GPR105 protein and the use of such compounds to control, prevent and/or treat conditions or diseases mediated by the GPR105 protein. The compounds of the present invention are useful for the treatment of diabetes, particularly Type 2 diabetes, hyperglycemia, insulin resistance, lipid disorders, obesity, atherosclerosis, and Metabolic Syndrome.

### BACKGROUND OF THE INVENTION

Metabolic Syndrome is a disorder that includes obesity, dyslipidaemia, and hyperglycemia. Metabolic Syndrome has increased to epidemic proportions worldwide. The pathophysiology of this syndrome is attributed to central distributed obesity, decreased high density lipoprotein, elevated triglycerides, elevated blood pressure and hyperglycemia. People suffering from Metabolic Syndrome are at increased risk of developing Type 2 diabetes, coronary heart disease, and other diseases related to plaque accumulation in artery walls (e.g., stroke and peripheral vascular disease). In two prospective European studies, Metabolic Syndrome was a predictor of increased cardiovascular disease and mortality (Isomaa et al., “Cardiovascular Morbidity and Mortality Associated With the Metabolic Syndrome,” Diabetes Care 24:683-689, 2001; Lakka et al., “The Metabolic Syndrome and Total and Cardiovascular Disease Mortality in Middle Aged Men,” JAMA 288:2709-2716, 2002).

The most significant underlying cause of Metabolic Syndrome is obesity. It has been disclosed in US 2007/0092913 (published on Apr. 26, 2007) that expression of GPR105 protein is correlated with weight gain and development of Type 2 diabetes. Furthermore, it has been demonstrated that antisense inhibition of GPR105 expression in mice reduces the rate at which the mice gain weight in response to a high fat diet. The mice also have lower levels of insulin, suggesting a decreased level of insulin resistance in these mice. Accordingly, GPR105 is a potential target for drugs that prevent diabetes, obesity or Metabolic Syndrome, or that ameliorate at least one symptom of Metabolic Syndrome.

The present invention provides a novel class of substituted fused pyrimidines as GPR105 antagonists which are useful for control, prevention, or treatment of obesity and diabetes, in particular, Type 2 diabetes and to ameliorate the symptoms of Metabolic Syndrome.

### SUMMARY OF THE INVENTION

The present invention relates to fused pyrimidine compounds of structural formula I:

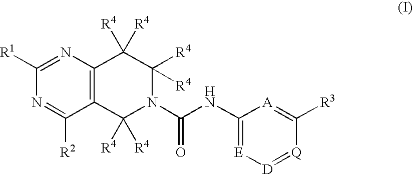

These fused pyrimidine compounds are effective as antagonists of the biological activity of the GPR105 protein. They are therefore useful for the treatment, control or prevention of disorders responsive to antagonism of this receptor, such as diabetes, in particular, Type 2 diabetes, hyperglycemia, insulin resistance, lipid disorders, obesity, atherosclerosis, and Metabolic Syndrome.

The present invention also relates to pharmaceutical compositions comprising the compounds of the present invention and a pharmaceutically acceptable carrier.

The present invention also relates to methods for the treatment, control, or prevention of disorders, diseases, or conditions responsive to antagonism of the GPR105 protein in a subject in need thereof by administering the compounds and pharmaceutical compositions of the present invention.

The present invention also relates to methods for the treatment, control, or prevention of diabetes, in particular, Type 2 diabetes, insulin resistance, obesity, lipid disorders, atherosclerosis, and Metabolic Syndrome by administering the compounds and pharmaceutical compositions of the present invention.

The present invention also relates to methods for the treatment, control, or prevention of obesity by administering the compounds of the present invention in combination with a therapeutically effective amount of another agent known to be useful to treat the condition.

The present invention also relates to methods for the treatment, control, or prevention of Type 2 diabetes by administering the compounds of the present invention in combination with a therapeutically effective amount of another agent known to be useful to treat the condition.

The present invention also relates to methods for the treatment, control, or prevention of atherosclerosis by administering the compounds of the present invention in combination with a therapeutically effective amount of another agent known to be useful to treat the condition.

The present invention also relates to methods for the treatment, control, or prevention of lipid disorders by administering the compounds of the present invention in combination with a therapeutically effective amount of another agent known to be useful to treat the condition.

The present invention also relates to methods for treating Metabolic Syndrome by administering the compounds of the present invention in combination with a therapeutically effective amount of another agent known to be useful to treat the condition.

### DETAILED DESCRIPTION OF THE INVENTION

The present invention relates to compounds of formula I:

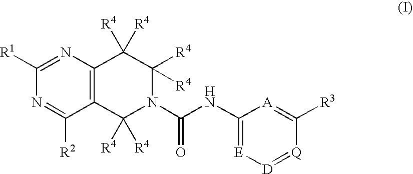

and pharmaceutically acceptable salts thereof, wherein
A, Q, D, and E are each independently N or CR⁸, with the proviso that at least two of A, Q, D, and E represent CR⁸;
R¹ is aryl or heteroaryl wherein aryl and heteroaryl are optionally substituted with one to three substituents independently selected from R^{a}:
R^{a} is selected from the group consisting of:
cyano,
halogen,
C₁₋₆ alkyl, optionally substituted with one hydroxy and one to six fluorines,
C₂₋₆ alkenyl,
C₂₋₆ alkynyl,
C₁₋₆ alkoxy, optionally substituted with one to five fluorines,
C₁₋₆ alkylthio, optionally substituted with one to five fluorines,
C₁₋₆ alkylsulfonyl, optionally substituted with one to five fluorines, (CH₂)ₙC₃₋₆ cycloalkyl, wherein cycloalkyl is optionally substituted with one to three substituents independently selected from halogen, hydroxy, cyano, nitro, CO₂H, C₁₋₆ alkyloxycarbonyl, C₁₋₆ alkyl, and C₁₋₆ alkoxy, wherein alkyl and alkoxy are optionally substituted with one to five fluorines,
(CH₂)ₙOR⁵,
(CH₂)ₙN(R⁵)₂,
(CH₂)ₙC≡N,
(CH₂)ₙCO₂R⁵,
(CH₂)ₙNR¹⁰SO₂R⁹,
(CH₂)ₙSO₂N(R⁵)₂,
(CH₂)ₙS(O)ᵣR⁵,
(CH₂)ₙNR¹⁰C(O)N(R⁵)₂,
(CH₂)ₙC(O)N(R⁵)₂,
(CH₂)ₙNR¹⁰C(O)R⁵,
(CH₂)ₙNR¹⁰CO₂R⁹,
(CH₂)ₙC(O)R⁵,
aryl, and
heteroaryl;
   wherein aryl and heteroaryl are optionally substituted with one to three substituents independently selected from the group consisting of halogen, C₁₋₄ alkyl, —CO₂C₁₋₄ alkyl, and CF₃ and wherein any individual methylene (CH₂) carbon atom in (CH₂)ₙ is optionally substituted with one to two substituents independently selected from fluorine, hydroxy, C₁₋₄ alkyl, and C₁₋₄ alkoxy, wherein alkyl and alkoxy are optionally substituted with one to five fluorines; or two substituents when on the same methylene (CH₂) group are taken together with the carbon atom to which they are attached to form a cyclopropyl group;

### R² is

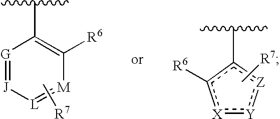

wherein R⁶ is selected from the group consisting of:
C₁₋₆ alkyl, optionally substituted with hydroxy, C₁₋₃ alkoxy, or one to five fluorines;
C₂₋₆ alkenyl,
C₂₋₆ alkynyl,
(CH₂)ₙ—C₃₋₆ cycloalkyl, wherein cycloalkyl is optionally substituted with one to three substituents independently selected from halogen, hydroxy, cyano, nitro, CO₂H, C₁₋₆ alkyloxycarbonyl, C₁₋₆ alkyl, and C₁₋₆ alkoxy, wherein alkyl and alkoxy are optionally substituted with one to five fluorines,
cyano,
halogen,
hydroxy,
C₁₋₄ alkoxy, optionally substituted with one to five fluorines, and
C₁₋₄ alkylthio, optionally substituted with one to five fluorines;
   wherein any individual methylene (CH₂) carbon atom in (CH₂)ₙ is optionally substituted with one to two substituents independently selected from fluorine, hydroxy, C₁₋₄ alkyl, and C₁₋₄ alkoxy, wherein alkyl and alkoxy are optionally substituted with one to five fluorines; or two substituents when on the same methylene (CH₂) group are taken together with the carbon atom to which they are attached to form a cyclopropyl group;
   G, J, L and M are each independently N or CR⁷, with the proviso that at least two of G, J, L and M represent CR⁷;
   X, Y, and Z are each independently O, S, or N, with the proviso that the combination of X, Y, and Z cannot represent more than one O or S;
   each R⁷ is independently selected from the group consisting of hydrogen, halogen, and C₁₋₄ alkyl optionally substituted with one to five fluorines;
   R³ is selected from the group consisting of:
cyano,
halogen,
C₁₋₆ alkyl, optionally substituted with one to five fluorines, C₂₋₆ alkenyl,
C₂₋₆ alkynyl,
C₁₋₆ alkoxy, optionally substituted with one to five fluorines,
C₁₋₆ alkylthio, optionally substituted with one to five fluorines,
C₁₋₆ alkylsulfonyl, optionally substituted with one to five fluorines,
(CH₂)ₙ—C₃₋₆ cycloalkyl, wherein cycloalkyl is optionally substituted with one to three substituents independently selected from halogen, hydroxy, cyano, nitro, CO₂H, C₁₋₆ alkyloxycarbonyl, C₁₋₆ alkyl, and C₁₋₆ alkoxy, wherein alkyl and alkoxy are optionally substituted with one to five fluorines,
(CH₂)ₙOR⁵,
(CH₂)ₙN(R⁵)₂,
(CH₂)ₙC≡N,
(CH₂)ₙCO₂R⁵,
(CH₂)ₙNR¹⁰SO₂R⁹,
(CH₂)ₙSO₂N(R⁵)₂,
(CH₂)ₙS(O)ᵣR⁵,
(CH₂)ₙNR¹⁰C(O)N(R⁵)₂,
(CH₂)ₙC(O)N(R⁵)₂,
(CH₂)ₙNR¹⁰C(O)R⁵,
(CH₂)ₙNR¹⁰CO₂R⁹,
(CH₂)ₙC(O)R⁵,
CH═CH-aryl,
(CH₂)ₚ—W—(CH₂)_{q}aryl, and
(CH₂)ₚ—W—(CH₂)_{q}-heteroaryl;
   wherein W is a bond, O, S(O)ᵣ, or NR¹⁰; aryl and heteroaryl are optionally substituted with one to three R^{a} substituents; and any individual methylene (CH₂) carbon atom in (CH₂)ₙ, (CH₂)ₚ, or (CH₂)_{q} is optionally substituted with one to two substituents independently selected from fluorine, hydroxy, C₁₋₄ alkyl, and C₁₋₄ alkoxy, wherein alkyl and alkoxy are optionally substituted with one to five fluorines; or two substituents when on the same methylene (CH₂) group are taken together with the carbon atom to which they are attached to form a cyclopropyl group;
   each R⁸ is selected from the group consisting of:
hydrogen,
cyano,
halogen,
C₁₋₆ alkyl, optionally substituted with one to five fluorines,
C₂₋₆ alkenyl,
C₂₋₆ alkynyl,
C₁₋₆ alkoxy, optionally substituted with one to five fluorines, C₁₋₆ alkylthio, optionally substituted with one to five fluorines, C₁₋₆ alkylsulfonyl, optionally substituted with one to five fluorines, CH₂)ₙ—C₃₋₆ cycloalkyl, wherein cycloalkyl is optionally substituted with one to three substituents independently selected from halogen, hydroxy, cyano, nitro, CO₂H, C₁₋₆ alkyloxycarbonyl, C₁₋₆ alkyl, and C₁₋₆ alkoxy, wherein alkyl and alkoxy are optionally substituted with one to five fluorines,
(CH₂)ₙOR⁵,
(CH₂)ₙN(R⁵)₂,
(CH₂)ₙC≡N,
(CH₂)ₙCO₂R⁵,
(CH₂)ₙNR¹⁰SO₂R⁹,
(CH₂)ₙSO₂N(R⁵)₂,
(CH₂)ₙS(O)ᵣR⁵,
(CH₂)ₙNR¹⁰C(O)N(R⁵)₂,
(CH₂)ₙC(O)N(R⁵)₂,
(CH₂)ₙNR¹⁰C(O)R⁵,
(CH₂)ₙNR¹⁰CO₂R⁹,
(CH₂)ₙC(O)R⁵,
(CH₂)ₚ—W—(CH₂)_{q}-aryl, and (CH₂)ₚ—W—(CH₂)_{q}-heteroaryl;
   wherein W is a bond, O, S(O)ᵣ, or NR¹⁰; aryl and heteroaryl are optionally substituted with one to three R^{a} substituents; and any individual methylene (CH₂) carbon atom in (CH₂)ₙ, (CH₂)ₚ, or (CH₂)_{q} is optionally substituted with one to two substituents independently selected from fluorine, hydroxy, C₁₋₄ alkyl, and C₁₋₄ alkoxy, wherein alkyl and alkoxy are optionally substituted with one to five fluorines; or two substituents when on the same methylene (CH₂) group are taken together with the carbon atom to which they are attached to form a cyclopropyl group;
   each R⁴ is independently hydrogen, fluorine, or C₁₋₃ alkyl; or two R⁴ groups together with the carbon atom to which they are attached can form a 3- to 6-membered carbocyclic ring system;
   each R⁵ is independently selected from the group consisting of
hydrogen,
C₁₋₆ alkyl, optionally substituted with one to five fluorines,
(CH₂)ₘ-aryl,
(CH₂)ₘ-heteroaryl, and
(CH₂)ₘC₃₋₆ cycloalkyl;
   wherein any individual methylene (CH₂) carbon atom in (CH₂)ₘ is optionally substituted with one to two substituents independently selected from fluorine, hydroxy, C₁₋₄ alkyl, and C₁₋₄ alkoxy, wherein alkyl and alkoxy are optionally substituted with one to five fluorines; or two substituents when on the same methylene (CH₂) group are taken together with the carbon atom to which they are attached to form a cyclopropyl group; and wherein alkyl, aryl, heteroaryl, and cycloalkyl are optionally substituted with one to three groups independently selected from halogen, C₁₋₄ alkyl, and C₁₋₄ alkoxy; or two R⁵ groups substituents together with the nitrogen atom to which they are attached form a heterocyclic ring selected from azetidine, pyrrolidine, piperidine, piperazine, and morpholine wherein said heterocyclic ring is unsubstituted or substituted with one to three substituents independently selected from halogen, hydroxy, C₁₋₆ alkyl, and C₁₋₆ alkoxy, wherein alkyl and alkoxy are optionally substituted with one to five fluorines;
   each R⁹ is independently C₁₋₆ alkyl, wherein alkyl is optionally substituted with one to five substituents independently selected from fluorine and hydroxy;
   R¹⁰ is hydrogen or R⁹;
   each n is independently an integer from 0 to 3;
   each m is independently an integer from 0 to 2;
   each p is an integer from 0 to 2;
   each q is an integer from 0 to 2; and
   each r is an integer from 0 to 2.

In one embodiment of the compounds of the present invention, R¹ is a phenyl group, a 5- or 6-membered monocyclic heteroaryl group, or a 9- or 10-membered bicyclic heteroaryl group containing one to three heteroatoms selected from O, S, and N, wherein the phenyl or heteroaryl group is optionally substituted with one to two substituents independently selected from R^{a}. In a class of this first embodiment, R¹ is a heteroaryl group selected from the group consisting of pyridinyl, N-oxo-pyridinyl, pyrimidinyl, isoxazolyl, thienyl, 1,3-benzodioxolyl, quinolyl, and pyrazolyl, each of which is optionally substituted with one to two substituents independently selected from R^{a}. In a subclass of this class, R¹ is pyridinyl or pyrimidinyl, each of which is optionally substituted with one to two substituents independently selected from R^{a}.

In a second class of this first embodiment, R¹ is phenyl optionally substituted with one to two substituents independently selected from R^{a}.

In a third class of this first embodiment, R^{a} is selected from the group consisting of halogen, C₁₋₃ alkyl, cyano, C₁₋₃ alkoxy, and —CO₂C₁₋₃ alkyl.

In a second embodiment of the compounds of the present invention, R² is

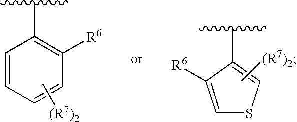

wherein R⁶ is selected from the group consisting of C₁₋₃ alkyl, chlorine, and bromine, and R⁷ is as defined above. In a class of this embodiment R⁶ is methyl or chlorine. In another class of this embodiment, R² is

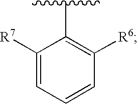

wherein is R⁶ is selected from the group consisting of C₁₋₃ alkyl, chlorine, and bromine, and R⁷ is as defined above. In a subclass of this class, R⁶ is methyl or chlorine. In a subclass of this subclass, R⁷ is hydrogen, methyl, chlorine, or fluorine. In another subclass of this subclass, R⁷ is hydrogen.

In a third embodiment of the compounds of the present invention, A and E are CH; D is N or CR⁸; and Q is CR⁸, wherein R⁸ is as defined above.

In one class of this third embodiment, D is N, and Q is CR⁸.

In a second class of this third embodiment, D is CH, and Q is CR⁸.

In a third class of this third embodiment, Q is CH, and D is CR⁸.

In a fourth class of this embodiment, R³ is selected from the group consisting of:
—CH₂—C₁₋₅ alkyl, wherein —CH₂— is optionally substituted with one to two fluorines and alkyl is optionally substituted with one to five fluorines,
—C₃₋₆ cycloalkyl,
—C₁₋₄ alkenyl,
—C₁₋₄ alkoxy, optionally substituted with one to five fluorines,
—C₁₋₄ alkylthio, optionally substituted with one to five fluorines,
—CH₂-aryl,
—CH₂CH₂-aryl,
—W-aryl, and
—W-heteroaryl;
wherein W is a bond, O, or S; and aryl and heteroaryl are optionally substituted with one to three R^{a} substituents.

In a subclass of this fourth class, R³ is ethyl, optionally substituted with one to five fluorines. In a subclass of this subclass, R⁸ is selected from the group consisting of:
hydrogen,
halogen,
cyano,
C₁₋₃ alkyl, optionally substituted with one to five fluorines,
C₃₋₅ cycloalkyl,
—W-phenyl, and
—W-heteroaryl;
   wherein W is a bond, O, or S; and aryl and heteroaryl are optionally substituted with one to three R^{a} substituents.

In another subclass of this fourth class, R³ is phenyl, optionally substituted with one to three R^{a} substituents.

In a fourth embodiment of the compounds of the present invention, A, E, and Q are CH; D is N or CR⁸; and R³ is selected from the group consisting of:
—CH₂—C₁₋₅ alkyl, wherein —CH₂— is optionally substituted with one to two fluorines and alkyl is optionally substituted with one to five fluorines,
—C₃₋₆ cycloalkyl,
—C₂₋₄ alkenyl,
—C₁₋₄ alkoxy, optionally substituted with one to five fluorines,
—C₁₋₄ alkylthio, optionally substituted with one to five fluorines,
—CH₂-aryl,
—CH₂CH₂-aryl,
—W-aryl, and
—W-heteroaryl;
wherein W is a bond, O, or S; and aryl and heteroaryl are optionally substituted with one to three R^{a} substituents.

In a class of this fourth embodiment, R³ is ethyl, optionally substituted with one to five fluorines. In a subclass of this class, R⁸ is selected from the group consisting of:
hydrogen,
halogen,
cyano,
C₁₋₃ alkyl, optionally substituted with one to five fluorines,
C₃₋₅ cycloalkyl,
—W-phenyl, and
—W-heteroaryl;
   wherein W is a bond, O, or S; and aryl and heteroaryl are optionally substituted with one to three R^{a} substituents.

In a fifth embodiment of the compounds of the present invention, A, E, and D are CH; Q is CR⁸; and R³ is selected from the group consisting of:
—CH₂—C₁₋₅ alkyl, wherein —CH₂— is optionally substituted with one to two fluorines and alkyl is optionally substituted with one to five fluorines,
—C₃₋₆ cycloalkyl,
—C₁₋₄ alkenyl,
—C₁₋₄ alkoxy, optionally substituted with one to five fluorines,
—C₁₋₄ alkylthio, optionally substituted with one to five fluorines,
—CH₂-aryl,
—CH₂CH₂-aryl,
—W-aryl, and
—W-heteroaryl;
wherein W is a bond, O, or S; and aryl and heteroaryl are optionally substituted with one to three R^{a} substituents.

In a class of this fifth embodiment, R³ is ethyl, optionally substituted with one to five fluorines. In a subclass of this class, R⁸ is selected from the group consisting of:
hydrogen,
halogen,
cyano,
C₁₋₃ alkyl, optionally substituted with one to five fluorines, C₃₋₅ cycloalkyl,
—W-phenyl, and
—W-heteroaryl;
   wherein W is a bond, O, or S; and aryl and heteroaryl are optionally substituted with one to three R^{a} substituents.

In a sixth embodiment of the compounds of the present invention,

R¹ is a phenyl group, a 5- or 6-membered monocyclic heteroaryl group, or a 9- or 10-membered bicyclic heteroaryl group containing one to three heteroatoms selected from O, S, and N, wherein the phenyl or heteroaryl group is optionally substituted with one to two substituents independently selected from R^{a};

### R² is

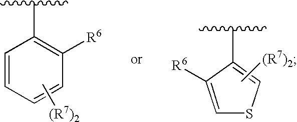

wherein R⁶ is selected from the group consisting of C₁₋₃ alkyl, chlorine, and bromine, and R⁷ is as defined above;

### A and E are CH;

### D is N or CR⁸;

### Q is CR⁸;

R³ is ethyl, optionally substituted with one to five fluorines; and
R⁸ is selected from the group consisting of:
hydrogen,
halogen,
cyano,
C₁₋₃ alkyl, optionally substituted with one to five fluorines,
C₃₋₅ cycloalkyl,
W-phenyl, and
—W-heteroaryl;
   wherein W is a bond, O, or S; and aryl and heteroaryl are optionally substituted with one to three R^{a} substituents. In a class of this embodiment, Q is CH.

Illustrative, but nonlimiting examples, of compounds of the present invention that are useful as antagonists of GPR105 activity are the following:

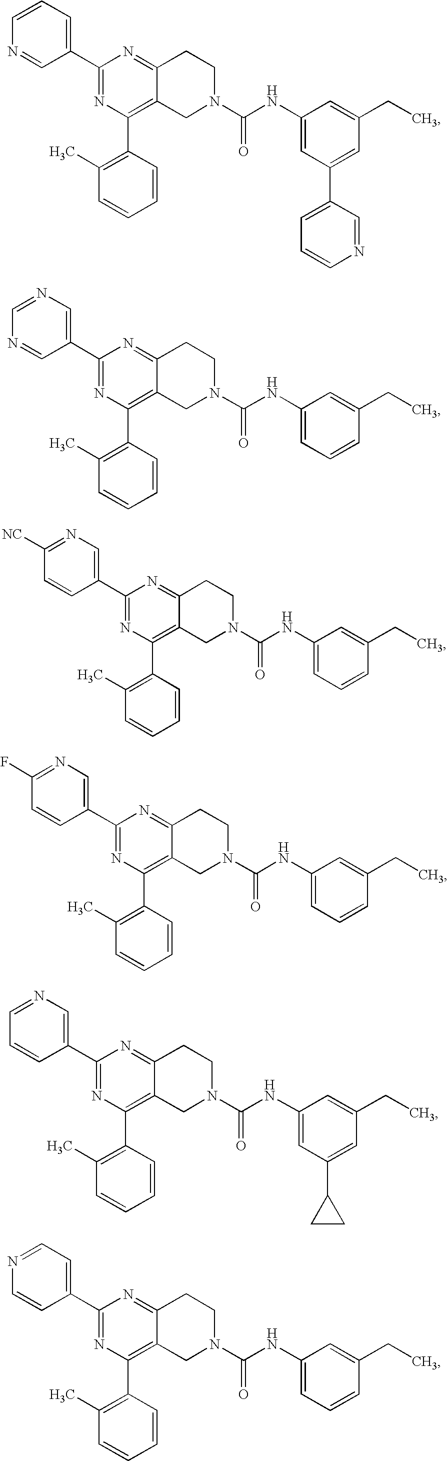

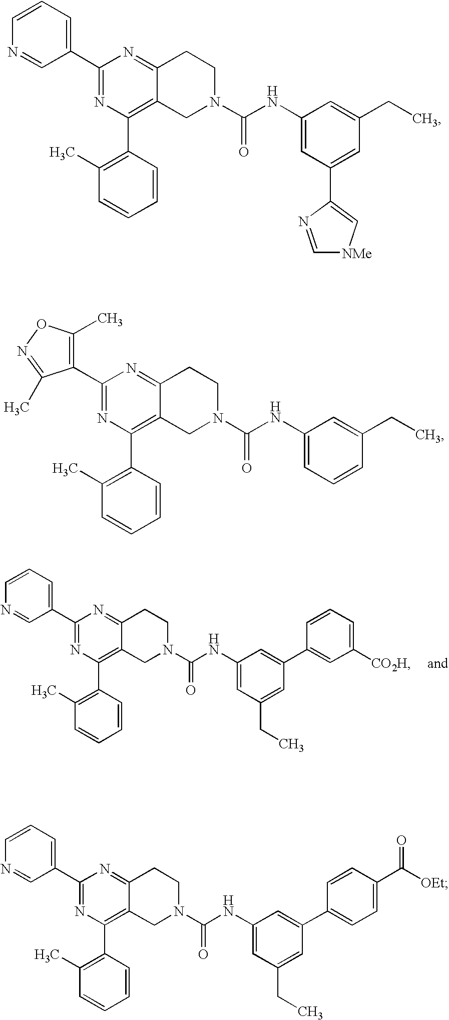

and pharmaceutically acceptable salts thereof.

As used herein the following definitions are applicable.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C₃₋₁₀, the term alkyl also includes cycloalkyl groups, and combinations of linear or branched alkyl chains combined with cycloalkyl structures. When no number of carbon atoms is specified, C₁₋₆ is intended.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkoxy” refers to straight or branched chain alkoxides of the number of carbon atoms specified (e.g., C₁₋₆ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₆ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₆ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂—), ethylsulfonyl, isopropylsulfonyl, etc.].

The term “alkylsulfinyl” refers to straight or branched chain alkylsulfoxides of the number of carbon atoms specified (e.g., C₁₋₆ alkylsulfinyl), or any number within this range [i.e., methylsulfinyl (MeSO—), ethylsulfinyl, isopropylsulfinyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO—), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, 2-oxopiperidin-1-yl, 2-oxopyrrolidin-1-yl, 2-oxoazetidin-1-yl, 1,2,4-oxadiazin-5(6H)-one-3-yl, and the like.

“Heteroaryl” means an aromatic or partially aromatic heterocycle that contains at least one ring heteroatom selected from O, S and N. Heteroaryls thus includes heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridinyl, N-oxo-pyridinyl, oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidinyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, 2-oxo-benzimidazolinyl, quinolyl, indolyl, isoquinolyl, dibenzofuranyl, 1,3-benzodioxolyl, imidazo[1,2-a]pyridinyl, [1,2,4-triazolo][4,3-a]pyridinyl, pyrazolo[1,5-c]pyridinyl, [1,2,4-triazolo][1,5-a]pyridinyl, 2-oxo-1,3-benzoxazolyl, 4-oxo-3H-quinazolinyl, 3-oxo-[1,2,4]-triazolo[4,3-a]-2H-pyridinyl, 5-oxo-[1,2,4]-4H-oxadiazolyl, 2-oxo-[1,3,4]-3H-oxadiazolyl, 2-oxo-1,3-dihydro-2H-imidazolyl, 3-oxo-2,4-dihydro-3H-1,2,4-triazolyl, 2,1,3-benzoxadiazolyl, and the like. For heterocyclyl and heteroaryl groups, rings and ring systems containing from 3-15 atoms are included, forming 1-3 rings. The atom of attachment of such heteroaryl group is either a carbon atom or a nitrogen where allowable by the rules of valency, such as pyrazol-1-yl and imidazol-1-yl.

“Halogen” refers to fluorine, chlorine, bromine and iodine.

### Optical Isomers-Diastereomers-Geometric Isomers-Tautomers:

Compounds of structural formula I may contain one or more asymmetric centers and can thus occur as racemates and racemic mixtures, single enantiomers, diastereomeric mixtures and individual diastereomers. The present invention is meant to comprehend all such isomeric forms of the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural formula I may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed with compounds of the present invention.

### Salts:

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethyl-aminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methyl-glucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula I are meant to also include the pharmaceutically acceptable salts.

### Dose Ranges:

The magnitude of prophylactic or therapeutic dose of a compound of Formula I will, of course, vary with the nature and severity of the condition to be treated, and with the particular compound of Formula I used and its route of administration. The dose will also vary according to the age, weight and response of the individual patient. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous administration is employed, a suitable dosage range is from about 0.01 mg to about 25 mg (preferably from 0.1 mg to about 10 mg) of a compound of Formula I per kg of body weight per day.

In the case where an oral composition is employed, a suitable dosage range is, e.g. from about 0.01 mg to about 100 mg of a compound of Formula I per kg of body weight per day, preferably from about 0.1 mg to about 10 mg per kg.

For use where a composition for sublingual administration is employed, a suitable dosage range is from 0.01 mg to about 25 mg (preferably from 0.1 mg to about 5 mg) of a compound of Formula I per kg of body weight per day.

### Pharmaceutical Compositions:

Another aspect of the present invention provides pharmaceutical compositions which comprises a compound of Formula I and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

Any suitable route of administration may be employed for providing a mammal, especially a human with an effective dosage of a compound of the present invention. For example, oral, sublingual, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of Formula I as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, sublingual, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulizers. The compounds may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of a compound of Formula I in suitable propellants, such as fluorocarbons or hydrocarbons and dry powder inhalation (DPI) aerosol, which may be formulated as a dry powder of a compound of Formula I with or without additional excipients.

Suitable topical formulations of a compound of formula I include transdermal devices, aerosols, creams, ointments, lotions, dusting powders, and the like.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 1 mg to about 500 mg of the active ingredient and each cachet or capsule contains from about 1 to about 500 mg of the active ingredient.

### Utilities and Combination Therapy:

The compounds of the present invention are useful for the control, prevention and treatment of conditions and diseases related to metabolic syndrome, including obesity, cardiovascular disease, such as atherosclerosis, diabetes, neurological disease, insulin resistance, cancer, and hepatic steatosis. The subject compounds are further useful in a method for the prevention or treatment of the aforementioned diseases, disorders and conditions in combination with other agents.

The compounds of the present invention may be used in combination with one or more other drugs in the treatment, prevention, suppression or amelioration of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also include therapies in which the compound of formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

Examples of other active ingredients that may be administered in combination with a compound of formula I, and either administered separately or in the same pharmaceutical composition, include, but are not limited to:

(a) dipeptidyl peptidase-IV (DPP-4) inhibitors;

(b) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, Galida, TAK-559, PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides, such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (N,N-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y1 or Y5 antagonists, CB1 receptor inverse agonists and antagonists, β3 adrenergic receptor agonists, melanocortin-receptor agonists, in particular melanocortin-4 receptor agonists, ghrelin antagonists, bombesin receptor agonists (such as bombesin receptor subtype-3 agonists), melanin-concentrating hormone (MCH) receptor antagonists, and inhibitors of microsomal triglyceride transfer protein;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib, and structures disclosed in WO 06/014413 and WO 06/014357;

(s) inhibitors of fructose 1,6-bisphosphatase, such as those disclosed in U.S. Pat. Nos. 6,054,587; 6,110,903; 6,284,748; 6,399,782; and 6,489,476;

(t) acetyl CoA carboxylase-1 and/or -2 inhibitors;

(u) AMPK activators;

(v) SCD1 inhibitors; and

(w) inhibitors of sodium-glucose co-transporter (SGLT-2).

Dipeptidyl peptidase-IV (DPP-4) inhibitors that can be combined with compounds of structural formula I include those disclosed in U.S. Pat. No. 6,699,871; WO 02/076450 (3 Oct. 2002); WO 03/004498 (16 Jan. 2003); WO 03/004496 (16 Jan. 2003); EP 1 258 476 (20 Nov. 2002); WO 02/083128 (24 Oct. 2002); WO 02/062764 (15 Aug. 2002); WO 03/000250 (3 Jan. 2003); WO 03/002530 (9 Jan. 2003); WO 03/002531 (9 Jan. 2003); WO 03/002553 (9 Jan. 2003); WO 03/002593 (9 Jan. 2003); WO 03/000180 (3 Jan. 2003); WO 03/082817 (9 Oct. 2003); WO 03/000181 (3 Jan. 2003); WO 04/007468 (22 Jan. 2004); WO 04/032836 (24 Apr. 2004); WO 04/037169 (6 May 2004); and WO 04/043940 (27 May 2004). Specific DPP-4 inhibitor compounds include sitagliptin (MK-0431); vildagliptin (LAF 237); denagliptin; P93/01; saxagliptin (BMS 477118); RO0730699; MP513; alogliptin (SYR-322); ABT-279; PHX1149; GRC-8200; and TS021.

Antiobesity compounds that can be combined with compounds of structural formula I include fenfluramine, dexfenfluramine, phentermine, sibutramine, orlistat, neuropeptide Y1 or Y5 antagonists, cannabinoid CB1 receptor antagonists or inverse agonists, melanocortin receptor agonists, in particular, melanocortin-4 receptor agonists, ghrelin antagonists, bombesin receptor agonists, and melanin-concentrating hormone (MCH) receptor antagonists. For a review of anti-obesity compounds that can be combined with compounds of structural formula I, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); and J. A. Fernandez-Lopez, et al., “Pharmacological Approaches for the Treatment of Obesity,” Drugs, 62: 915-944 (2002).

Neuropeptide Y5 antagonists that can be combined with compounds of structural formula I include those disclosed in U.S. Pat. No. 6,335,345 (1 Jan. 2002) and WO 01/14376 (1 Mar. 2001); and specific compounds identified as GW 59884A; GW 569180A; LY366377; and CGP-71683A.

Cannabinoid CB1 receptor antagonists that can be combined with compounds of formula I include those disclosed in U.S. Pat. No. 6,972,295, such as taranabant; U.S. Pat. No. 5,624,941, such as rimonabant; PCT Publication WO 02/076949, such as SLV-319; U.S. Pat. No. 6,028,084; PCT Publication WO 98/41519; PCT Publication WO 00/10968; PCT Publication WO 99/02499; U.S. Pat. No. 5,532,237; U.S. Pat. No. 5,292,736; PCT Publication WO 03/086288; PCT Publication WO 03/087037; PCT Publication WO 04/048317; PCT Publication WO 03/007887; PCT Publication WO 03/063781; PCT Publication WO 03/075660; PCT Publication WO 03/077847; PCT Publication WO 03/082190; PCT Publication WO 03/082191; PCT Publication WO 03/087037; PCT Publication WO 03/086288; PCT Publication WO 04/012671; PCT Publication WO 04/029204; PCT Publication WO 04/040040; PCT Publication WO 01/64632; PCT Publication WO 01/64633; and PCT Publication WO 01/64634.

Melanocortin-4 receptor (MC4R) agonists useful in the present invention include, but are not limited to, those disclosed in U.S. Pat. No. 6,294,534, U.S. Pat. Nos. 6,350,760, 6,376,509, 6,410,548, 6,458,790, U.S. Pat. No. 6,472,398, U.S. Pat. No. 5,837,521, U.S. Pat. No. 6,699,873, which are hereby incorporated by reference in their entirety; in US Patent Application Publication Nos. US 2002/0004512, US2002/0019523, US2002/0137664, US2003/0236262, US2003/0225060, US2003/0092732, US2003/109556, US 2002/0177151, US 2002/187932, US 2003/0113263, which are hereby incorporated by reference in their entirety; and in WO 99/64002, WO 00/74679, WO 02/15909, WO 01/70708, WO 01/70337, WO 01/91752, WO 02/068387, WO 02/068388, WO 02/067869, WO 03/007949, WO 2004/024720, WO 2004/089307, WO 2004/078716, WO 2004/078717, WO 2004/037797, WO 01/58891, WO 02/070511, WO 02/079146, WO 03/009847, WO 03/057671, WO 03/068738, WO 03/092690, WO 02/059095, WO 02/059107, WO 02/059108, WO 02/059117, WO 02/085925, WO 03/004480, WO 03/009850, WO 03/013571, WO 03/031410, WO 03/053927, WO 03/061660, WO 03/066597, WO 03/094918, WO 03/099818, WO 04/037797, WO 04/048345, WO 02/018327, WO 02/080896, WO 02/081443, WO 03/066587, WO 03/066597, WO 03/099818, WO 02/062766, WO 03/000663, WO 03/000666, WO 03/003977, WO 03/040107, WO 03/040117, WO 03/040118, WO 03/013509, WO 03/057671, WO 02/079753, WO 02/092566, WO 03/-093234, WO 03/095474, and WO 03/104761.

One particular aspect of combination therapy concerns a method of treating a condition selected from the group consisting of hypercholesterolemia, atherosclerosis, low HDL levels, high LDL levels, hyperlipidemia, hypertriglyceridemia, and dyslipidemia, in a mammalian patient in need of such treatment comprising administering to the patient a therapeutically effective amount of a compound of structural formula I and an HMG-CoA reductase inhibitor.

More particularly, this aspect of combination therapy concerns a method of treating a condition selected from the group consisting of hypercholesterolemia, atherosclerosis, low HDL levels, high LDL levels, hyperlipidemia, hypertriglyceridemia and dyslipidemia in a mammalian patient in need of such treatment wherein the HMG-CoA reductase inhibitor is a statin selected from the group consisting of lovastatin, simvastatin, pravastatin, cerivastatin, fluvastatin, atorvastatin, and rosuvastatin.

In another aspect of the invention, a method of reducing the risk of developing a condition selected from the group consisting of hypercholesterolemia, atherosclerosis, low HDL levels, high LDL levels, hyperlipidemia, hypertriglyceridemia and dyslipidemia, and the sequelae of such conditions is disclosed comprising administering to a mammalian patient in need of such treatment a therapeutically effective amount of a compound of structural formula I and an HMG-CoA reductase inhibitor.

In another aspect of the invention, a method for delaying the onset or reducing the risk of developing atherosclerosis in a human patient in need of such treatment is disclosed comprising administering to said patient an effective amount of a compound of structural formula I and an HMG-CoA reductase inhibitor.

More particularly, a method for delaying the onset or reducing the risk of developing atherosclerosis in a human patient in need of such treatment is disclosed, wherein the HMG-CoA reductase inhibitor is a statin selected from the group consisting of: lovastatin, simvastatin, pravastatin, cerivastatin, fluvastatin, atorvastatin, and rosuvastatin.

In another aspect of the invention, a method for delaying the onset or reducing the risk of developing atherosclerosis in a human patient in need of such treatment is disclosed,

wherein the HMG-Co A reductase inhibitor is a statin and further comprising administering a cholesterol absorption inhibitor.

More particularly, in another aspect of the invention, a method for delaying the onset or reducing the risk of developing atherosclerosis in a human patient in need of such treatment is disclosed, wherein the HMG-Co A reductase inhibitor is a statin and the cholesterol absorption inhibitor is ezetimibe.

In another aspect of the invention, a pharmaceutical composition is disclosed which comprises:

(1) a compound of structural formula I;
(2) a compound selected from the group consisting of:

(a) dipeptidyl peptidase-IV (DPP-4) inhibitors;

(b) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, Galida, TAK-559, PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (N,N-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y1 or Y5 antagonists, CB1 receptor inverse agonists and antagonists, β3 adrenergic receptor agonists, melanocortin-receptor agonists, in particular melanocortin-4 receptor agonists, ghrelin antagonists, bombesin receptor agonists (such as bombesin receptor subtype-3 agonists), melanin-concentrating hormone (MCH) receptor antagonists, and inhibitors of microsomal triglyceride transfer protein;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib, and structures disclosed in WO 06/014413 and WO 06/014357;

(s) inhibitors of fructose 1,6-bisphosphatase, such as those disclosed in U.S. Pat. Nos. 6,054,587; 6,110,903; 6,284,748; 6,399,782; and 6,489,476;

(t) acetyl CoA carboxylase-1 and/or -2 inhibitors; and

(u) AMPK activators;

(v) SCD1 inhibitors; and

(w) inhibitors of sodium-glucose co-transporter (SGLT-2); and

(3) a pharmaceutically acceptable carrier.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s)

### Assays for Determining Biological Activity:

### A. Cell-Binding Assay:

A stable HEK clonal cell line expressing the chimpanzee GPR105 protein and the chimeric G protein Gqi5 was developed. The chimeric Gqi5 forces the coupling of GPR105 through the Gq (calcium) pathway and allows for monitoring of calcium signaling using a calcium binding fluorescent dye and the FLIPR (fluorometric imaging plate reader, MDS Sciex). 12,500 HEK/GPR105/Gqi5 expressing cells were plated in 25 μL Dulbecco's Modified Eagle's Medium (DMEM) containing 10% fetal bovine serum (FBS) onto 384-well, poly-D-lysine coated plates. Cells were incubated overnight at 37° C. and 5% CO₂ to form a monolayer. On the following day, 30 μL of fluorescent no-wash dye was added to the cell monolayer and the plate was incubated for 60 min at 37° C., 5% CO₂. 250 mL of compound in 100% DMSO was added to cell/dye incubation using acoustic dispensing (Echo™, Labcyte). Following a 20 minute incubation of compound at room temperature, 6.25 μL of UDP-glucose agonist (at EC₈₀) in Hank's Balanced Salt Solution (HBSS) containing 20 mM Hepes was added to cells and Ca²⁺ signaling was monitored by FLIPR. Quantitation of the % inhibition of Ca²⁺ signaling by antagonist was calculated using the maximum fluorescent signal detected. IC₅₀'s for the compounds of structural formula (I) were calculated as follows:

a.) Basal=incubation of cells+DMSO+Buffer;
b.) ECK)=incubation of cells of DMSO+agonist to achieve 80% maximum stimulation of calcium release;
c.) Compound=incubation of cells+antagonist in DMSO+ECH agonist;
d.) Calcium release monitored by Fluorescence (RFU relative fluorescence units) using the FLIPR;
e.) The percentage of inhibition was calculated according to the equation: (1−(compound sample−Basal)/(EC80−Basal))×100;
f.) The percentage of inhibition at each dose was plotted, the Four Parameter Logistic Fit performed to draw the curve and the IC₅₀ is the compound dose where the % inhibition=50%.

The compounds of structural formula I, particularly the compounds of Examples 1 through 7 and Examples listed in Table 2 below, exhibit an inhibition constant IC₅₀ of less than 1 micromolar (μM) and more typically less than 500 nanomolar (nM). Representative inhibition IC₅₀'s for compounds of the present invention against the chimpanzee GPR105 protein are provided in Table 1:

TABLE 1

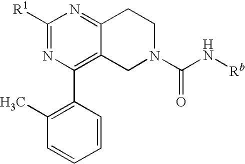

| | | IC₅₀ |
|---|---|---|
| R¹ | R^{b} | (nM) |
| 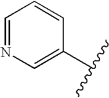 | 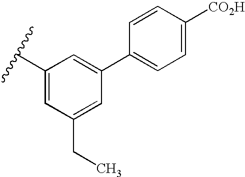 | 4.0 |
| 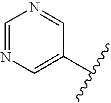 | 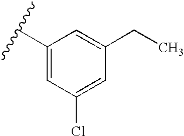 | 10.5 |
| 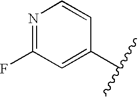 | 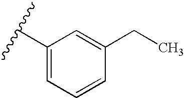 | 12.0 |
| 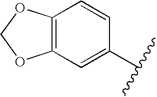 | 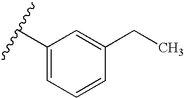 | 88.5 |
| 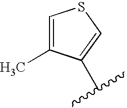 | 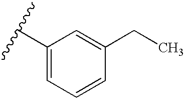 | 65.1 |
| 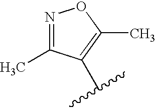 | 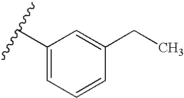 | 4.4 |
| 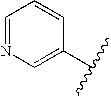 | 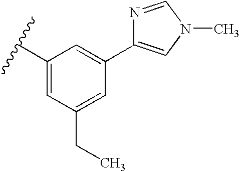 | 5.6 |
| 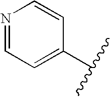 | 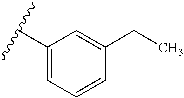 | 6.7 |
| 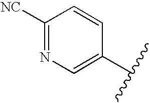 | 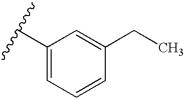 | 7.8 |
| 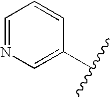 | 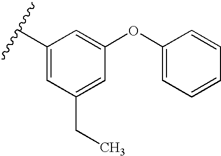 | 8.1 |
| 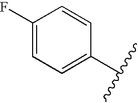 | 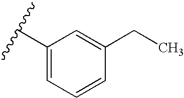 | 98.5 |
| 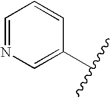 | 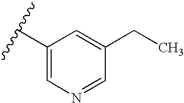 | 55.7 |
| 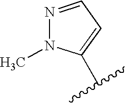 | 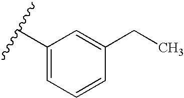 | 34.5 |
| 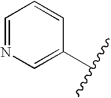 | 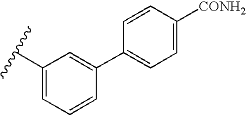 | 18.4 |

### B. Diet-Induced Obese [DIO] Mouse Protocol

a. Established DIO [eDIO]

C57B1/6 mice at 6 weeks of age are placed on a high fat diet [Research Diets D12492] consisting of fat, carbohydrate and protein at 60:20:20 kcal %. Mice of at least 20 weeks of age [14 weeks on the high fat diet] are used for the experiments. One week before compound treatment, the mice are dosed orally with the study vehicle to acclimate the mice with the dosing procedure [mock dosing]. A test compound or the vehicle is then administered orally either once or twice daily for a two-week period. Body weight, food consumption, and plasma compound levels from a satellite group of mice are measured at regular intervals during the study period. In this paradigm, loss of body weight from an established obesity state is the target endpoint. At the end of the study, additional endpoints such as plasma insulin, leptin, adiponectin levels, plasma glucose, blood lipid profile, blood cell counts and tissue compound levels are measured as needed.

b. Growing DIO [gDIO]

The protocol is similar to that used for eDIO mice except that mock dosing followed by compound treatment is given to young growing mice at 6-7 weeks of age at the same time when they are fed with the high fat diet. In this case, prevention of body weight gain is measured. Terminal endpoints as listed above are obtained as appropriate.

### Methods of Synthesis of the Compounds of Structural Formula (I):

The compounds of structural formula I can be prepared according to the procedures of the following Schemes and Examples, using appropriate materials and are further exemplified by the following specific examples. The compounds illustrated in the examples are not, however, to be construed as forming the only genus that is considered as the invention. The Examples further illustrate details for the preparation of the compounds of the present invention. Those skilled in the art will readily understand that known variations of protecting groups, as well as of the conditions and processes of the following preparative procedures, can be used to prepare these compounds. It is also understood that whenever a chemical reagent such as an isocyanate, a boronic acid, or a boronate is not commercially available, such a chemical reagent can be readily prepared following one of numerous methods described in the literature. All temperatures are degrees Celsius unless otherwise noted. Mass spectra (MS) were measured either by electrospray ion-mass spectroscopy (ESMS) or by atmospheric pressure chemical ionization mass spectroscopy (APCI).

### LIST OF ABBREVIATIONS

Alk=alkyl
APCI=atmospheric pressure chemical ionization
Ar=aryl
Boc=tert-butoxycarbonyl
br=broad
Cbz=benzyloxycarbonyl
CH₂Cl₂=dichloromethane
d=doublet

### DIPEA=N,N-diisopropylethylamine

DMAP=4-dimethylaminopyridine

### DMF=N,N-dimethylformamide

DMSO=dimethylsulfoxide
ESI=electrospray ionization
EtOAc=ethyl acetate
h=hour(s)
HOAc=acetic acid
KOH=potassium hydroxide
LC-MS=liquid chromatography-mass spectroscopy
LiOH=lithium hydroxide
m=multiplet
min=minutes
MeOH=methyl alcohol
MgSO₄=magnesium sulfate
MS=mass spectroscopy
NaOH=sodium hydroxide
Na₂SO₄=sodium sulfate
NH₄OAc=ammonium acetate
NMR=nuclear magnetic resonance spectroscopy
PG=protecting group
rt=room temperature
s=singlet
t=triplet
THF=tetrahydrofuran
TFA=trifluoroacetic acid
TLC=thin-layer chromatography
TsCl=p-toluenesulfonyl chloride

### Method A (Scheme 1):

The commercial available keto-ester 1 is reacted with an appropriate isocyanate to give urea 2. Reaction of urea 2 with an appropriately substituted amidine and a base in an alcoholic solvent provides the 3-hydroxypyrimidine 3, which can be converted to tosylate 4 under standard conditions. Suzuki coupling of 4 with an appropriately substituted boronic acid yields final product 5.

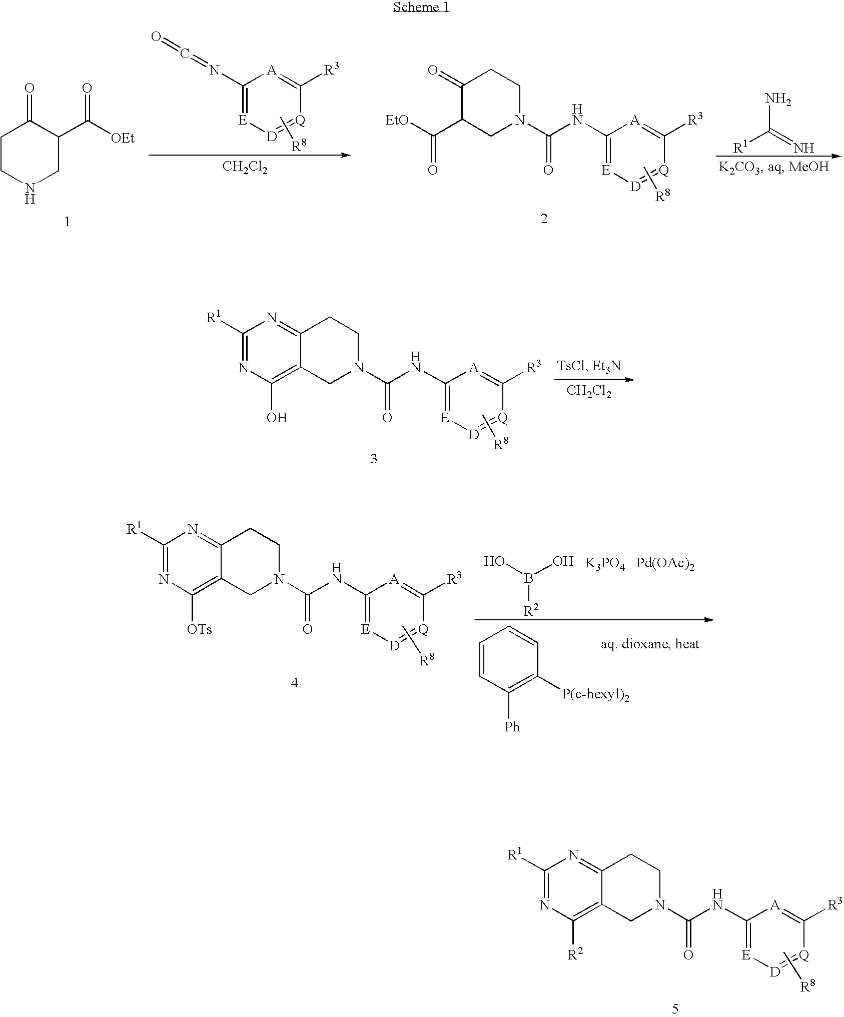

### Method B (Scheme 2):

The commercial available keto-ester 1 can be protected as its Boc derivative 6. Following the same reaction sequence as described in Method A, intermediate 6 can be converted to intermediate 9. Cleavage of the Boc group under acidic conditions provides amine 10, which can be reacted with an appropriate isocyanate to afford product 5.

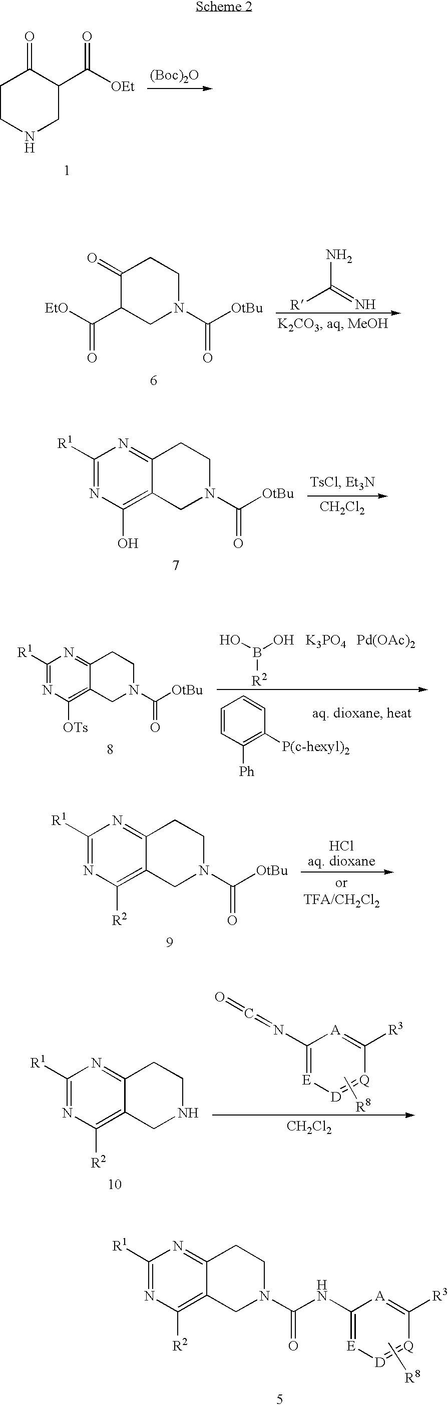

### Method C (Scheme 3):

Reaction of keto-ester 6 with guanidinium chloride and a base provides intermediate 11. The hydroxy group in 11 can be selectively tosylated to give tosylate 12, which can undergo a Suzuki cross-coupling reaction with an appropriately substituted boronic acid to yield 13. Following Glaser's procedure (J. Am. Chem. Soc., 2005, Vol. 127, pages 880-887), intermediate 13 can be converted to bromide 14. Removal of the Boc group in 14 with dry HCl/dioxane results in displacement of the bromine atom by chloride to give hydrochloride salt 15. Reaction of 15 with an appropriate isocyanate affords urea 16. Suzuki coupling of 16 with the choice of a boronic acid yields product 5.

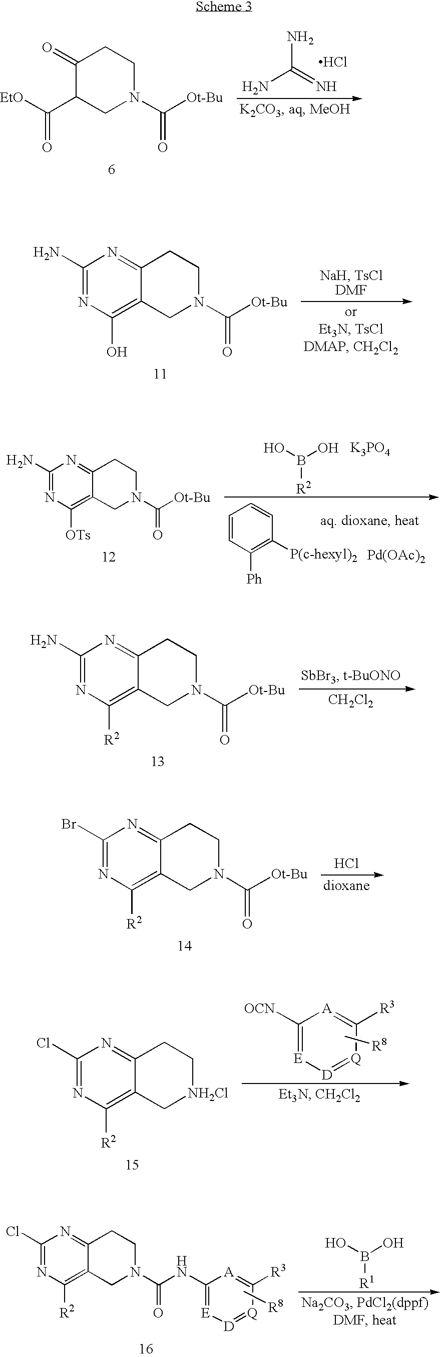

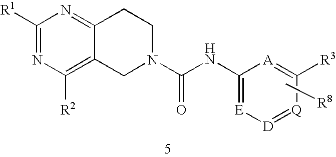

### Method D (Scheme 4):

Treatment of intermediate 6 with O-methylisourea and base provides intermediate 17. Following the same reaction sequence as described in Method A, intermediate 17 can be converted to intermediate 19. Dealkylation of the ether with concomitant removal of the Boc protecting group provides a hydroxy amine intermediate, which can be reacted with an appropriate isocyanate to afford hydroxy-pyrimidine 20. Chlorination using phosphoryl chloride affords compound 16 which is further transformed into 5 as described in Method C.

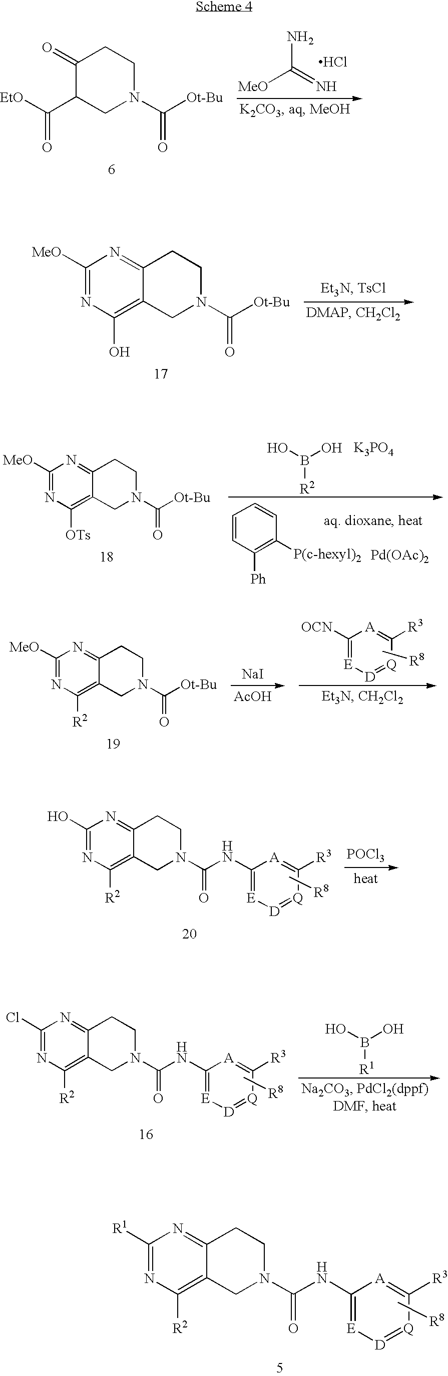

### Method E (Scheme 5):

When R⁸ is a group such as Cl, Br or OTs as in 5′, metal-catalyzed cross-coupling reactions, such as Suzuki or Stille reactions, as well as other types of cross-coupling reactions, such as the modified Ullmann-type diaryl ether synthesis described in Organic Letters, Vol. 5, pages 3799-3802 (2003), can be used to further elaborate the structure and obtain final compounds of structural formula 6.

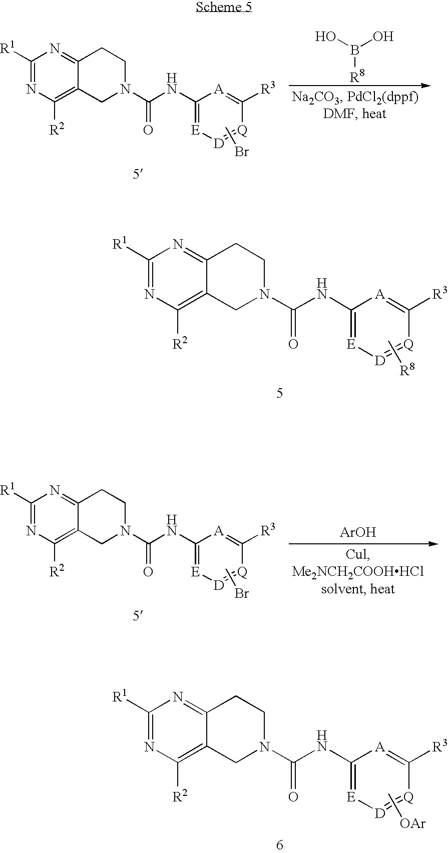

### Example 1

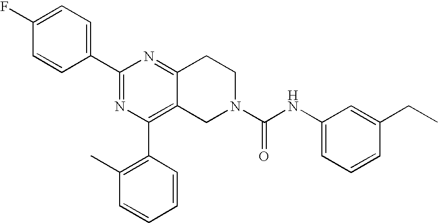

### N-(3-Ethylphenyl)-2-(4-fluorophenyl)-4-(2-methylphenyl)-7,8-dihydropyrido[4,3-d]pyrimidine-6(5H)-carboxamide

### Step 1: tert-Butyl 2-amino-4-hydroxy-7,8-dihydropyrido[4,3-d]pyrimidine-6(5H)-carboxylate

A suspension of 1-tert-butyl 3-ethyl 4-oxopiperidine-1,3-dicarboxylate (65.3 g, 241 mmol), guanidine hydrochloride (23 g, 241 mmol) and potassium carbonate (66.5 g, 481 mmol) in water (180 mL) and methanol (120 mL) was stirred at 70° overnight. The reaction mixture was poured into hydrochloric acid (2 N) and the pH adjusted to 7. The title compound was collected by filtration, triturated with ether, filtered, and dried to give the title compound as a white solid. MS (+ESI): m/z 266.9 (M⁺H).

### Step 2: tert-Butyl 2-amino-4-{[(4-methylphenyl)sulfonyl]oxy}-7,8-dihydropyrido[4,3-d]pyrimidine-6(5H)-carboxylate

To a stirred suspension of the product of Step 1 (58 g, 218 mmol) in dichloromethane (600 mL) at room temperature were added triethylamine (36.4 mL, 261 mmol), DMAP (2.66 g, 21.8 mmol) followed by a solution of p-toluenesulfonyl chloride (41.5 g, 218 mmol) in dichloromethane (300 mL). The reaction mixture was stirred at room temperature overnight. Water (1000 mL) was added, and the mixture was filtered. The filtrate was partitioned and extracted twice with 10% methanol-dichloromethane (600 mL). The combined organic layers were dried with MgSO₄ and concentrated under vacuum. The residue was purified by column chromatography on silica gel eluting with ethyl acetate/hexanes (40:60 to 100:0) to afford the title compound.

### Step 3: tert-Butyl 2-amino-4-(2-methylphenyl)-7,8-dihydropyrido[4,3-d]pyrimidine-6(5H)-carboxylate

To a stirred solution of the product of Step 2 (36 g, 86 mmol) and 2-methylphenylboronic acid (34.9 g, 257 mmol) in dioxane (2 L) and water (20 mL) at room temperature were added K₃PO₄ (109 g, 514 mmol) and 2-(dicyclohexylphosphino)biphenyl (9.00 g, 25.7 mmol). The reaction mixture was bubbled with nitrogen for 10 min, then palladium(II) acetate (2.88 g, 12.84 mmol) was added, and bubbled again with nitrogen for 10 min. The reaction mixture was stirred at 80° overnight. The reaction mixture was poured into aqueous sodium hydrogen carbonate and ethyl acetate and filtered through Celite. It was then partitioned and the aqueous layer extracted twice with ethyl acetate (600 mL). The combined organic layers were washed with brine, dried with MgSO₄ and concentrated under vacuum. The residue was purified by column chromatography on silica gel eluting with ethyl acetate/hexanes (40:60 to 100:0).

### Step 4: tert-Butyl 2-bromo-4-(2-methylphenyl)-7,8-dihydropyrido[4,3-d]pyrimidine-6(5H)-carboxylate

To a stirred suspension of the product of Step 3 (8.9 g, 26.1 mmol) and antimony(III) bromide (6.8 mL, 78 mmol) in dibromomethane (330 mL) at −10° was added dropwise tert-butyl nitrite (22 mL, 185 mmol). The reaction mixture was warmed up to 0° and aged for 2 h. It was then stirred at 0° overnight and then poured into aqueous sodium hydrogen carbonate-ice-dichloromethane, filtered through Celite and rinsed with dichloromethane. The filtrate was partitioned and extracted twice with dichloromethane. The combined organic layers were washed with brine, dried with MgSO₄ and concentrated under vacuum. The residue was purified by column chromatography on silica gel eluting with ethyl acetate/hexanes (10:90 to 100:0) to give the title compound.

### Step 5: 2-Chloro-4-(2-methylphenyl)-5,6,7,8-tetrahydropyrido[4,3-d]pyrimidin-6-ium chloride

A mixture of the product of Step 4 (5 g, 12.4 mmol) in hydrochloric acid in dioxane (56 mL, 224 mmol) was stirred at room temperature for 3 h. It was then concentrated to dryness and the residue was used as such in the next step.

### Step 6: 2-Chloro-N-(3-ethylphenyl)-4-(2-methylphenyl)-7,8-dihydropyrido[4,3-d]pyrimidine-6(5H)-carboxamide

To a stirred solution of the product of Step 5 (3.66 g, 12.4 mmol) and triethylamine (3.5 mL, 25 mmol) in dichloromethane (82 mL) at room temperature was added dropwise 3-ethylphenyl isocyanate (1.8 mL, 12.4 mmol). The reaction mixture was stirred at room temperature for 2 h. It was then poured into water and extracted twice with dichloromethane (150 mL). The combined organic layers were washed with brine, dried with MgSO₄ and concentrated under vacuum. The residue was purified by column chromatography on silica gel eluting with acetonitrile/dichloromethane (0:100 to 10:90) to give the desired compound.

### Step 7: N-(3-Ethylphenyl)-2-(4-fluorophenyl)-4-(2-methylphenyl)-7,8-dihydropyrido[4,3-d]pyrimidine-6(5H)-carboxamide

To a stirred solution of the product of Step 6 (200 mg, 0.49 mmol) and 4-fluorophenylboronic acid (83 mg, 0.59 mmol) in DMF (3 mL) at room temperature was added sodium carbonate (0.61 mL, 1.2 mmol). The reaction mixture was bubbled with nitrogen for 10 min, then PdCl₂(dppf) (36.0 mg, 0.05 mmol) was added, bubbled again with nitrogen for 10 min. The reaction mixture was stirred at 90° for 3 h. It was then poured into aqueous sodium hydrogen carbonate and extracted twice with ethyl acetate (60 mL). The combined organic layers were washed three times with water, brine, dried with MgSO₄ and concentrated under vacuum. The residue was purified by column chromatography on silica gel using automated gradient pump system CombiFlash eluting with ethyl acetate/hexanes (10:90 to 30:70 for 20 min, then at 30:70 for 5 min) to give the title compound as a colorless foam. MS (+ESI): m/z 467.1 (M⁺H).

### Example 2

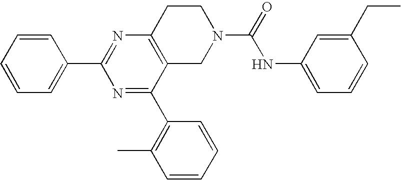

### N-(3-Ethylphenyl)-4-(2-methylphenyl)-2-phenyl-7,8-dihydropyrido[4,3-d]pyrimidine-6(5H)-carboxamide

### Step 1: 1-tert-Butyl 3-ethyl 4-oxopiperidine-1,3-dicarboxylate

To a solution of ethyl 4-piperidone-3-carboxylate hydrochloride (25 g, 120 mmol), triethylamine (50.6 mL, 360 mmol) in THF (605 mL) at room temperature was added di-tert-butyl dicarbonate (28.1 mL, 120 mmol) portionwise and the mixture was stirred at room temperature for 3 h. The reaction was quenched with aqueous ammonium chloride at room temperature and was extracted with ethyl acetate (700 mL). The combined organic fractions were washed with brine, dried (Na₂SO₄), filtered and the solvent was evaporated to afford the crude desired product. ¹H NMR (400 MHz, acetone-d₆): δ 4.31-4.13 (m, 2H), 4.06 (s, 2H), 3.71-3.57 (m, 2H), 2.57-2.33 (m, 2H), 1.49 (s, 9H), 1.30 (dt, 3H).

### Step 2: tert-Butyl 4-hydroxy-2-phenyl-7,8-dihydropyrido[4,3-d]pyrimidine-6(5H)-carboxylate

To a suspension of benzenecarboximidamide hydrochloride (5 g, 31.9 mmol), 1-tert-butyl 3-ethyl 4-oxopiperidine-1,3-dicarboxylate (8.66 g, 31.9 mmol) in water (60 mL) were added MeOH (40 mL) and potassium carbonate (8.82 g, 63.9 mmol) at room temperature and the mixture was stirred at 70° overnight. The reaction was quenched with hydrochloric acid (2M) at 0° C. to adjust the pH to around 7.5 and was then filtered to collect the solid. The solid thus obtained was dissolved in dichloromethane, dried (Na₂SO₄), filtered and the solvent was evaporated. The residue was triturated with ether and the solid was collected by filtration to afford the desired product as a white solid.

### Step 3: tert-Butyl 4-{[(4-methylphenyl)sulfonyl]oxy}-2-phenyl-7,8-dihydropyrido[4,3-d]pyrimidine-6(5H)-carboxylate

To a solution of tert-butyl 4-hydroxy-2-phenyl-7,8-dihydropyrido[4,3-d]pyrimidine-6(5H)-carboxylate (8.8 g, 26.9 mmol) and triethylamine (3.75 mL, 26.9 mmol) was added DMAP (0.328 g, 2.69 mmol) in CH₂Cl₂ (300 mL) followed by p-toluenesulfonyl chloride (5.12 g, 26.9 mmol) portionwise at room temperature and the mixture was stirred at room temperature for 1 h. The reaction was then quenched with aqueous sodium hydrogen carbonate at room temperature and was extracted three times with dichloromethane (100 mL). The combined organic extracts were washed with brine, dried (Na₂SO₄), filtered and the solvent was evaporated to yield the desired compound.

### Step 4: tert-Butyl 4-(2-methylphenyl)-2-phenyl-7,8-dihydropyrido[4,3-d]pyrimidine-6(5H)-carboxylate

A suspension of tert-butyl 4-{[(4-methylphenyl)sulfonyl]oxy}-2-phenyl-7,8-dihydropyrido[4,3-d]pyrimidine-6(5H)-carboxylate (12 g, 24.92 mmol), palladium(II) acetate (0.559 g, 2.49 mmol), 2-(dicyclohexylphosphino)biphenyl (1.75 g, 4.98 mmol), potassium phosphate, tribasic (15.9 mL, 74.8 mmol), and 2-methylphenylboronic acid (6.78 g, 49.8 mmol) in dioxane (249 mL) was heated at 80° overnight. After cooling to room temperature, the reaction mixture was diluted with EtOAc, filtered through a silica gel pad, and the filtrate was concentrated. The residue was purified by column chromatography on silica gel eluting with 10% EtOAc/hexane to give the title compound as a white foam.

### Step 5: 4-(2-Methylphenyl)-2-phenyl-5,6,7,8-tetrahydropyrido[4,3-d]pyrimidine dihydrochloride

A mixture of tert-butyl 4-(2-methylphenyl)-2-phenyl-7,8-dihydropyrido[4,3-d]pyrimidine-6(5H)-carboxylate (8.8 g, 21.9 mmol) and hydrochloric acid in 1,4-dioxane (99 mL, 395 mmol) was stirred at room temperature for 3 h. The mixture was diluted with hexane and the solid was collected by filtration to afford the title compound.

### Step 6: N-(3-Ethylphenyl)-4-(2-methylphenyl)-2-phenyl-7,8-dihydropyrido[4,3-d]pyrimidine-6(5H)-carboxamide

To a solution of: 4-(2-methylphenyl)-2-phenyl-5,6,7,8-tetrahydropyrido[4,3-d]pyrimidine dihydrochloride (1 g, 3.3 mmol) and triethylamine (0.92 mL, 6.6 mmol) in CH₂Cl₂ (20 mL) at room temperature was added 3-ethylphenyl isocyanate (0.47 mL, 3.3 mmol) dropwise and the mixture was stirred at room temperature for 1 h. The solvent was removed and the residue was diluted with toluene and filtered. The solution was loaded onto a silica gel column and eluted with 0-80% EtOAc/hexane to afford N-(3-ethylphenyl)-4-(2-methylphenyl)-2-phenyl-7,8-dihydropyrido[4,3-d]pyrimidine-6(5H)-carboxamide. ¹H NMR (500 MHz, acetone-d₆): δ 8.51-8.48 (m, 2H), 8.13 (s, 1H), 7.52-7.27 (m, 8H), 7.12 (t, 1H), 6.82 (d, 1H), 4.52 (s, 2H), 4.01 (s, 2H), 3.19 (t, 2H), 2.57 (q, 2H), 2.23 (s, 3H), 1.20 (t, 3H). MS (+ESI) m/z 449.1 (M⁺H).

### Example 3

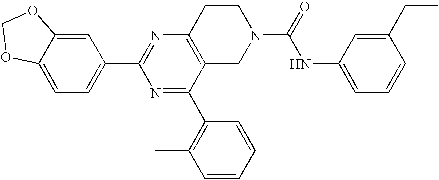

### 2-(1,3-Benzodioxole-5-yl)-N-(3-ethylphenyl)-4-(2-methylphenyl)-7,8-dihydropyrido[4,3-d]pyrimidine-6(5H)-carboxamide

### Step 1: Ethyl 1-{[(3-ethylphenyl)amino]carbonyl}-4-oxopiperidine-3-carboxylate

To a solution of ethyl 4-oxopiperidine-3-carboxylate (6.01 g, 35.1 mmol) in dichloromethane at room temperature was added 3-ethylphenyl isocyanate (5 mL, 35.1 mmol) and the mixture was stirred for 3 h at room temperature. The reaction mixture was then loaded on a silica gel column and purified by eluting with 0-100% EtOAc/hexane to afford ethyl 1-{[(3-ethylphenyl)amino]carbonyl}-4-oxopiperidine-3-carboxylate as a white powder.

### Step 2: 1,3-Benzodioxole-5-carboximidamide hydrochloride

To a solution of lithium bis(trimethylsilyl)amide in THF (238 mL, 238 mmol) at 0° was added 1,3-benzodioxole-5-carbonitrile (35 g, 238 mmol) portionwise under a N₂ flow and the mixture was stirred at room temperature for 6 h. The mixture was then cooled down to −78° and 150 mL of 6 N HCl in isopropanol/water was added at a speed such that the internal temperature did not rise above 0°. The mixture was stirred at 0° for 0.5 h and filtered. The solid was then washed with ether and dried under vacuum at 50° to give 1,3-benzodioxole-5-carboximidamide hydrochloride as a yellowish solid.

### Step 3: 2-(1,3-Benzodioxole-5-yl)-N-(3-ethylphenyl)-4-hydroxy-7,8-dihydropyrido[4,3-d]pyrimidine-6(5H)-carboxamide

Ethyl 1-{[(3-ethylphenyl)amino]carbonyl}-4-oxopiperidine-3-carboxylate (40 g, 126 mmol) and 1,3-benzodioxole-5-carboximidamide hydrochloride (25.2 g, 126 mmol) were dissolved in MeOH (300 mL) by gentle heating. To this mixture, water (360 mL) and potassium carbonate (43.4 g, 314 mmol) were added and the mixture was stirred at 65° for 7 h. The reaction was quenched by slow addition of acetic acid (28.8 mL). The solid was collected by filtration and dried at 50° under vacuum to provide the title compound as a white solid.

### Step 4: 2(1,3-Benzodioxole-5-yl)-6-{[(3-ethylphenyl)amino]carbonyl}-5,6,7,8-tetrahydropyrido[4,3-d]pyrimidin-4-yl 4-methylbenzenesulfonate

To a suspension of 2-(1,3-benzodioxole-5-yl)-N-(3-ethylphenyl)-4-hydroxy-7,8-dihydropyrido[4,3-d]pyrimidine-6(5H)-carboxamide (14 g, 33.5 mmol), triethylamine (5.60 mL, 40.1 mmol), and DMAP (0.409 g, 3.35 mmol) in CH₂Cl₂ at room temperature was added p-toluenesulfonyl chloride (7.02 g, 36.8 mmol) portionwise and the mixture was stirred at room temperature for 6 h. The reaction mixture was then at reflux temperature for 45 min. The mixture was treated with 15 mL of water and the mixture was stirred at room temperature for 30 min. Solid Na₂SO₄ was added and the mixture was filtered. The solvent was evaporated and the residue was triturated with ether to afford a solid that was filtered and dried to provide the title compound.

### Step 5: 2-(1,3-Benzodioxole-5-yl)-N-(3-ethylphenyl)-4-(2-methylphenyl)-7,8-dihydropyrido[4,3-d]pyrimidine-6(5H)-carboxamide

To a mixture of 2-(1,3-benzodioxole-5-yl)-6-{[(3-ethylphenyl)amino]carbonyl}-5,6,7,8-tetrahydropyrido[4,3-d]pyrimidin-4-yl 4-methylbenzenesulfonate (19.2 g, 33.5 mmol), potassium phosphate, tribasic (42.6 mL, 201 mmol), palladium(II) acetate (1.13 g, 5.02 mmol), 2-(dicyclohexylphosphino)biphenyl (3.52 g, 10.0 mmol), and 2-methylphenylboronic acid (15.9 g, 117 mmol) under nitrogen, a mixture of 1,4-dioxane (1000 mL) and water (10.00 mL) was added. The resulted mixture was heated to 85° for 12 h. The mixture was then treated slowly with 50 mL of 8 N KOH and then stirred at rt for 30 min. Hexane (800 mL) was introduced and Na₂SO₄ was added to solidify the aqueous phase. The resulting mixture was passed through a silica gel pad and washed with EtOAc. After evaporating the solvent, the residue was purified by flash chromatography on silica gel (0-60% EtOAc/hexane) to afford the desired product which contained small amount of 2-methylphenylboronic acid. The material was dissolved in dichloromethane and washed with 100 mL of 1 N KOH and dried. After evaporation of the solvent, the residue was purified again on a silica gel column eluted with 0-60% EtOAc/hexane to afford the title compound as a white solid. ¹H NMR (500 MHz, acetone-d₆): δ 8.12-8.10 (m, 2H), 7.94 (d, 1H), 7.47-7.27 (m, 6H), 7.12 (t, 1H), 6.96 (d, 1H), 6.81 (d, 1H), 6.10 (s, 2H), 4.49 (s, 2H), 3.99 (s, 2H), 3.16 (t, 2H), 2.57 (q, 2H), 2.23 (s, 3H), 1.18 (t, 3H). MS (+ESI): m/z 493.2 (M⁺H).

### Example 4

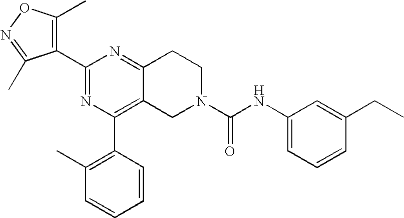

### 2-(3,5-Dimethylisozaxol-4-yl)-N-(3-ethylphenyl)-4-(2-methylphenyl)-7,8-dihydropyrido[4,3-d]pyrimidine-6(5H)-carboxamide

### Step 1: tert-Butyl 4-hydroxy-2-methoxy-7,8-dihydropyrido[4,3-d]pyrimidine-6(5H)-carboxylate

1-tert-Butyl 3-ethyl-4-oxopiperidine-1,3-dicarboxylate (32.7 g, 121 mmol), O-methylisourea hydrochloride (13.32 g, 121 mmol) and potassium carbonate (33.3 g, 241 mmol) were heated together in a mixture of water (90 mL) and methanol (60 mL) at 70° C. overnight. Following completion of the reaction, the mixture was cooled to rt, poured into water and the mixture was adjusted to pH 7 with 2N HCl. The solid was collected by filtration, washed with water, dried under suction and finally triturated with ether to afford tert-butyl 4-hydroxy-2-methoxy-7,8-dihydropyrido[4,3-d]pyrimidine-6(5H)-carboxylate as a white powder.

### Step 2: tert-Butyl 2-methoxy-4-{[(4-methylphenyl)sulfonyl]oxy}-7,8-dihydropyrido[4,3-d]pyrimidine-6-(5H)-carboxylate

A solution of DMAP (0.929 g, 7.61 mmol), p-toluenesulfonyl chloride (16.0 g, 84.0 mmol) and tert-butyl 4-hydroxy-2-methoxy-7,8-dihydropyrido[4,3-d]pyrimidine-6(5H)-carboxylate (21.4 g, 76.0 mmol) in dichloromethane (150 mL) was treated dropwise with triethylamine (12.7 mL, 91.0 mmol) with stirring at rt for 4 h. The mixture was then poured into water and extracted twice with dichloromethane (400 mL). The combined extracts were washed with aqueous ammonium chloride and water, and dried over MgSO₄. Concentration in vacuo gave tert-butyl-2-methoxy-4-{[(4-methylphenyl)sulfonyl]oxy}-7,8-dihydropyrido[4,3-d]pyrimidine-6-(5H)-carboxylate as a brown gum.

### Step 3: tert-Butyl 2-methoxy-4-(2-methylphenyl)-7,8-dihydropyrido[4,3-d]pyrimidine-6-(5H)-carboxylate

Nitrogen gas was bubbled for 10 min into a mixture of tert-butyl 2-methoxy-4-{[(4-methylphenyl)sulfonyl]oxy}-7,8-dihydropyrido[4,3-d]pyrimidine-6-(5H)-carboxylate (31.0 g, 71.2 mmol), o-tolylboronic acid (12.1 g, 89.0 mmol), K₃PO₄ (91.0 g, 430 mmol) and palladium(II) acetate (2.34 g, 10.7 mmol) in DMF (300 mL). (Dicyclohexylphosphino)biphenyl (7.48 g, 21.4 mmol) was then added and the mixture was heated at 80° under a nitrogen atmosphere for 16 h. The vessel contents were then partitioned between aqueous sodium bicarbonate solution and ethyl acetate. The layers were separated and the aqueous phase was extracted with additional ethyl acetate (100 mL). The combined organics were washed with brine, dried over MgSO₄ and concentrated. Flash chromatography of the residue on silica gel eluting with ethyl acetate/hexanes (1:3 to 1:2) provided tert-butyl-2-methoxy-4-(2-methylphenyl)-7,8-dihydropyrido[4,3-d]pyrimidine-6-(5H)-carboxylate as a yellow gum.

### Step 4: N-(3-ethylphenyl)-2-hydroxy-4-(2-methylphenyl)-7,8-dihydro[4,3-d]pyrimidine-6(5H)-carboxamide

Sodium iodide (5.06 g, 33.8 mmol) was added to a solution of tert-butyl 2-methoxy-4-(2-methylphenyl)-7,8-dihydropyrido[4,3-d]pyrimidine-6-(5H)-carboxylate (8.0 g, 22 mmol) in acetic acid (90 mL) with heating at 110° for 16 h. The acetic acid was removed by rotary evaporation under high vacuum followed by two coevaporations of the residue with toluene and heating at 50° under high vacuum overnight. This material was then suspended in dichloromethane (100 mL) and treated with triethylamine (9.4 mL, 68 mmol) with stirring at 0° for 15 min 3-Ethylphenyl isocyanate (3.2 mL, 22 mmol) was subsequently introduced with stirring at rt overnight. The reaction mixture was then partitioned between dichloromethane and aqueous ammonium chloride solution that was adjusted to pH 6 with HCl. The organic phase was washed with water, dried over MgSO₄ and concentrated. Flash chromatography on silica gel eluting first with ethyl acetate/hexanes (3/7), then with methanol/dichloromethane (5/95 to 10/90) gave N-(3-ethylphenyl)-2-hydroxy-4-(2-methylphenyl)-7,8-dihydro[4,3-d]pyrimidine-6(5H)-carboxamide as a white solid.

### Step 5: 2-Chloro-N-(3-ethylphenyl)-4-(2-methylphenyl)-7,8-dihydropyrido[4,3-d]pyrimidine-6(5H)-carboxamide

N-(3-Ethylphenyl)-2-hydroxy-4-(2-methylphenyl)-7,8-dihydro[4,3-d]pyrimidine-6(5H)-carboxamide and POCl₃ (32 mL, 340 mmol) were heated together at 100° for 3 h. The mixture was then concentrated to dryness, diluted with dichloromethane (250 mL) and the solution was stirred vigorously with saturated aqueous sodium bicarbonate overnight at rt. The layers were separated and the organic phase was washed with water, dried over MgSO₄ and concentrated. The residue was subjected to flash chromatography on silica gel eluting with CH₃CN/CH₂Cl₂ (0:100 to 10:90) to afford 2-chloro-N-(3-ethylphenyl)-4-(2-methylphenyl)-7,8-dihydropyrido[4,3-d]pyrimidine-6(5H)-carboxamide as a yellow solid. MS (+ESI): m/z 407.1, 409.1 (M⁺H).

### Step 6: 2-(3,5-Dimethylisozaxol-4-yl)-N-(3-ethylphenyl)-4-(2-methylphenyl)-7,8-dihydropyrido[4,3-d]pyrimidine-6(5H)-carboxamide

Nitrogen gas was bubbled for 10 min into a mixture of 2-chloro-N-(3-ethylphenyl)-4-(2-methylphenyl)-7,8-dihydropyrido[4,3-d]pyrimidine-6(5H)-carboxamide (60 mg, 0.15 mmol), (3,5-dimethylisoxazol-4-yl)boronic acid (31 mg, 0.22 mmol), aqueous sodium carbonate (2 M, 0.18 mL, 0.37 mmol), Pd(dppf)Cl₂.CH₂Cl₂ (11 mg, 0.015 mmol) and DMF (1.5 mL). The mixture was then stirred at 90° under a nitrogen atmosphere. After 4 h at this temperature, the reaction vessel contents were cooled to rt and partitioned between aqueous sodium bicarbonate solution and ethyl acetate. The layers were separated and the aqueous phase was extracted with additional ethyl acetate. The combined organics were washed with brine, dried over MgSO₄ and concentrated. Flash chromatography of the residue on silica gel eluting with ethyl acetate/hexanes (1:4 to 45:55) gave the title compound as a white solid.

MS (+ESI): m/z 468.3 (M⁺H).

### Example 5

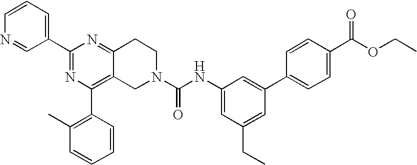

### Ethyl 3′-ethyl-5′-({[4-(2-methylphenyl)-2-pyridin-3-yl-7,8-dihydropyrido[4,3-d]pyrimidin-6(5H)-yl]carbonyl}amino)biphenyl-4-carboxylate

To N-(3-bromo-5-ethylphenyl)-4-(2-methylphenyl)-2-pyridin-3-yl-7,8-dihydropyrido[4,3-d]pyrimidine-6(5H)-carboxamide in a 20:1 mixture of toluene/water (0.1M) were added tricyclohexylphosphine (0.1 eq), potassium phosphate tribasic (4 eq), palladium(II) acetate (0.05 eq) and 4-ethoxycarbonylphenylboronic acid (2 eq). Nitrogen was bubbled into the mixture for 5 min and the reaction flask was sealed and heated to 100° for 12 h. The reaction was cooled to rt and diluted with brine. The reaction mixture was extracted with EtOAc, the combined organic layers were washed with water and brine, dried over Na₂SO₄, filtered and concentrated. The crude product was purified by Combiflash chromatography on silica gel using a solvent gradient of 20-50% CH₃CN/CH₂Cl₂ to afford the title compound. MS (+ESI): m/z 598.4 (M⁺H).

### Example 6

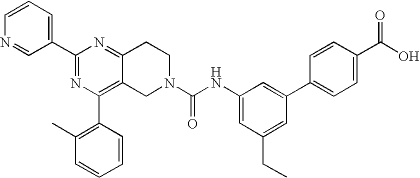

### 3′-Ethyl-5′-({[4-(2-methylphenyl)-2-pyridin-3-yl-7,8-dihydropyrido[4,3-d]pyrimidin-6(5H)-yl]carbonyl}amino)biphenyl-4-carboxylic acid

To ethyl 3′-ethyl-5′-({[4-(2-methylphenyl)-2-pyridin-3-yl-7,8-dihydropyrido[4,3-d]pyrimidin-6(5H)-yl]carbonyl}amino)biphenyl-4-carboxylate in a 3:1 mixture of THF/MeOH (0.03M) was added 1N aqueous LiOH (5 eq) and the reaction was stirred at rt for 6 h. The reaction mixture was acidified to pH 5-6 with AcOH and was concentrated under vacuum. The residue was suspended in water and the resulting white solid was filtered, washed with water and ether and dried under high vacuum to afford title compound. MS (+ESI): m/z 570.2 (M⁺H).

### Example 7

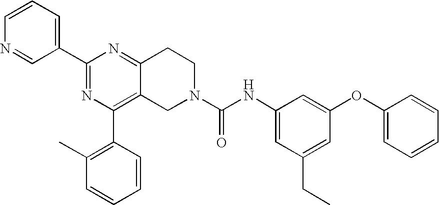

### N-(3-Ethyl-5-phenoxyphenyl)-4-(2-methylphenyl)-2-pyridin-3-yl-7,8-dihydropyrido[4,3-d]pyrimidine-6(5H)-carboxamide

To N-(3-bromo-5-ethylphenyl)-4-(2-methylphenyl)-2-pyridin-3-yl-7,8-dihydropyrido[4,3-d]pyrimidine-6(5H)-carboxamide in dioxane (0.28 M) were added cesium carbonate (2 eq), copper(I) iodide (0.1 eq), N,N-dimethylglycine hydrochloride (0.3 eq) and phenol (1.5 eq). Nitrogen was bubbled into the mixture for 5 min and the reaction flask was sealed and heated to 90° for 12 h. The reaction was cooled to rt and diluted with brine. The reaction mixture was extracted with EtOAc, the combined organic layers were washed with water and brine, dried over Na₂SO₄, filtered and concentrated. The crude product was purified by Combiflash chromatography on silica gel using a solvent gradient of 20-60% CH₃CN/CH₂Cl₂ to afford the title compound. MS (+ESI): m/z 542.0 (M⁺H).

The additional Examples listed in Table 2 were prepared following essentially the procedures outlined for Examples 1-7 as shown in Schemes 1-5.

TABLE 2

| | | MS |
|---|---|---|
| | | (ESI, M + |
| Example | Structure | H) |
| 8 | 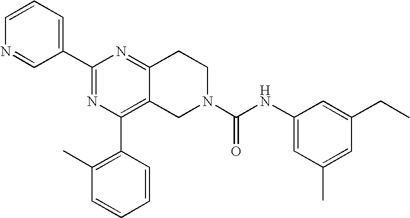 | 464.2 |
| 9 | 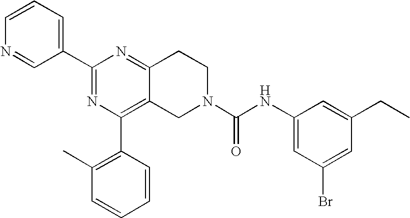 | 530.0 |
| 10 | 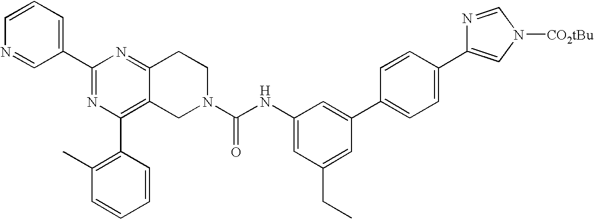 | 692.3 |
| 11 | 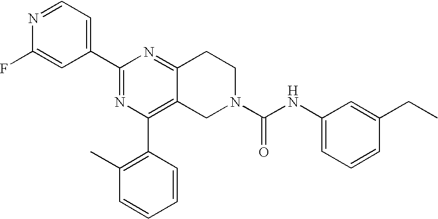 | 468.1 |
| 12 | 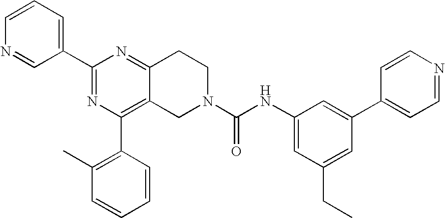 | 527.0 |
| 13 | 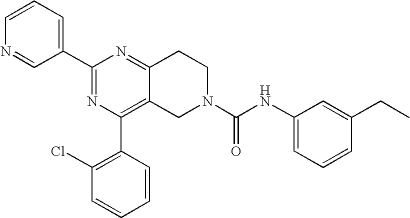 | 470.2 |
| 14 | 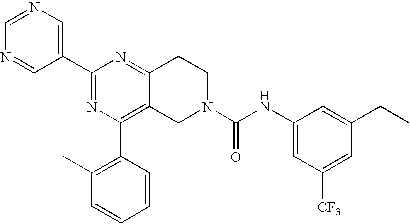 | 519.2 |
| 15 | 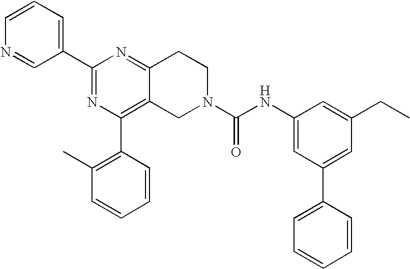 | 526.0 |
| 16 | 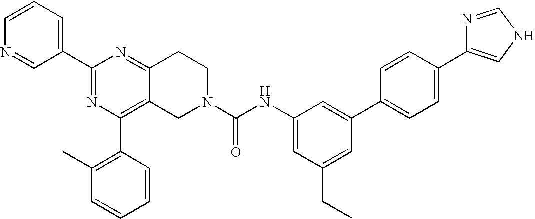 | 592.5 |
| 17 | 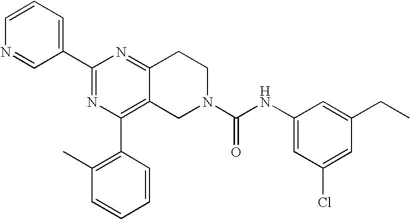 | 486.0 |
| 18 | 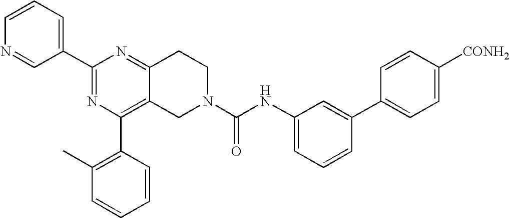 | 541.3 |
| 19 | 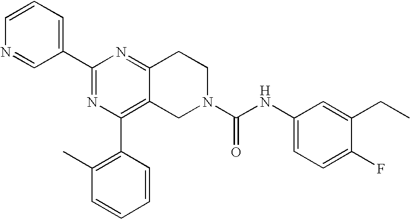 | 468.1 |
| 20 | 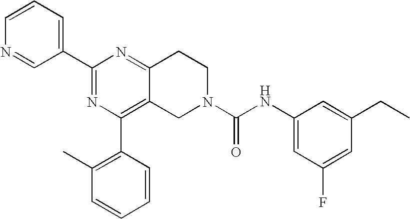 | 468.1 |
| 21 | 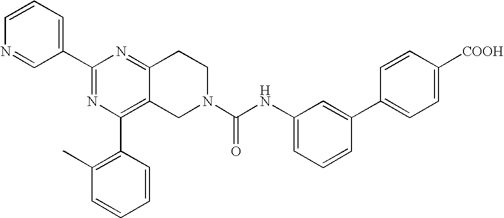 | 542.1 |
| 22 | 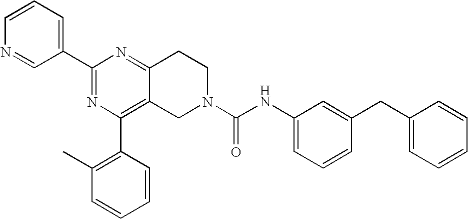 | 512.1 |
| 23 | 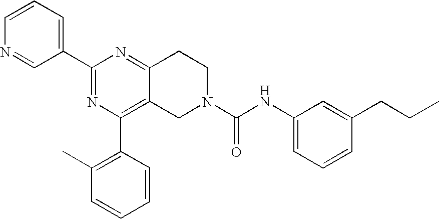 | 464.3 |
| 24 | 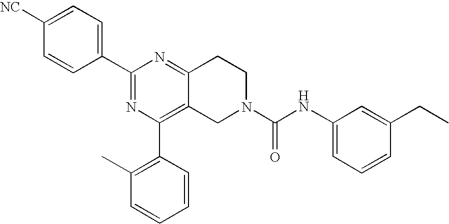 | 474.1 |
| 25 | 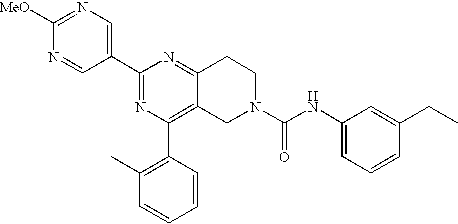 | 481.2 |
| 26 | 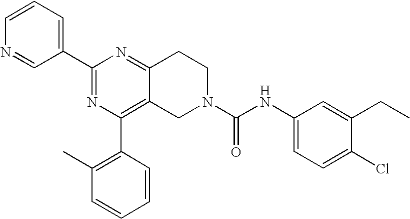 | 486.0 |
| 27 | 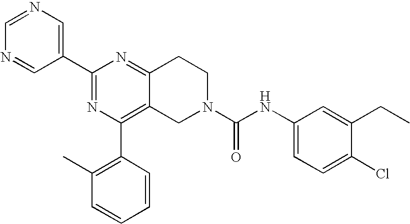 | 485.2 |
| 28 | 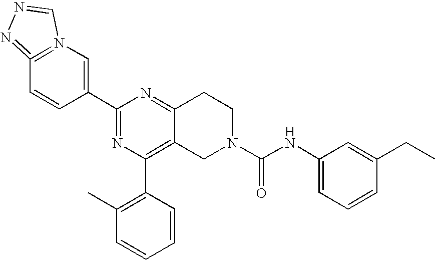 | 490.1 |
| 29 | 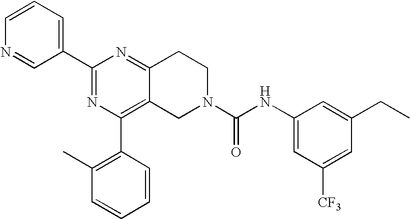 | 518.3 |
| 30 | 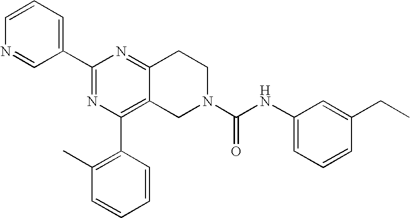 | 450.1 |
| 31 | 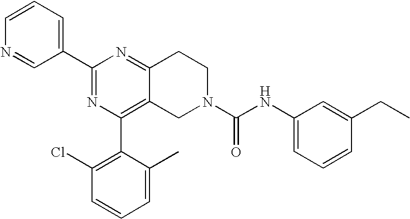 | 486.1 |
| 32 | 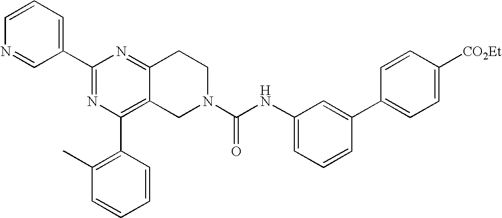 | 570.2 |
| 33 | 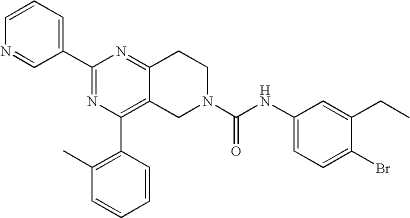 | 530.0 |
| 34 | 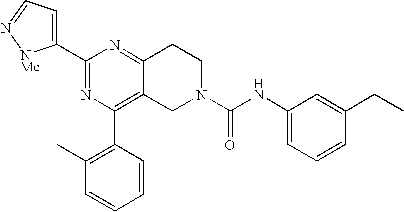 | 453.1 |
| 35 | 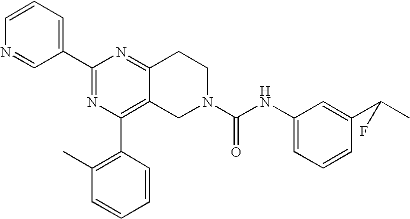 | 468.0 |
| 36 | 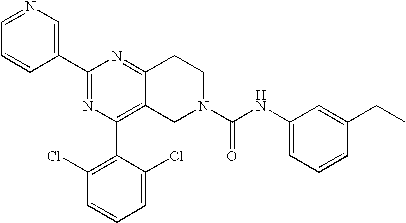 | 506.0 |
| 37 | 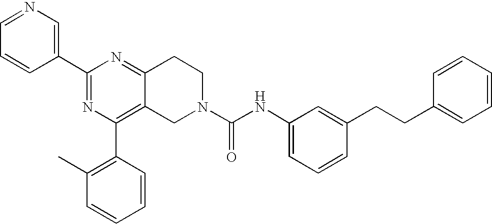 | 526.2 |
| 38 | 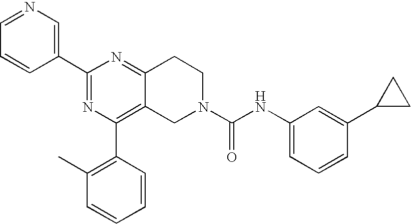 | 461.7 |
| 39 | 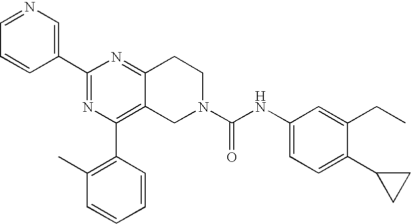 | 490.1 |
| 40 | 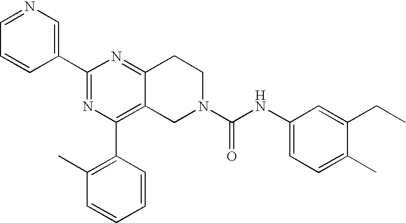 | 464.1 |
| 41 | 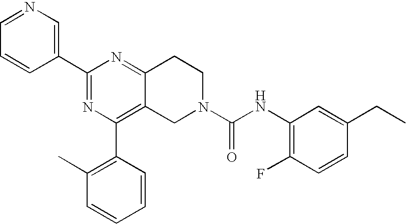 | 468.4 |
| 42 | 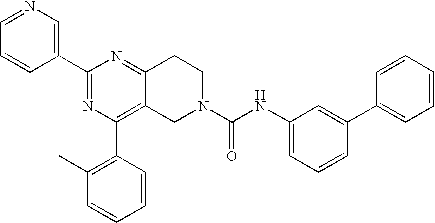 | 498.1 |
| 43 | 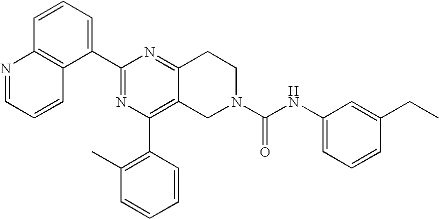 | 500.1 |
| 44 | 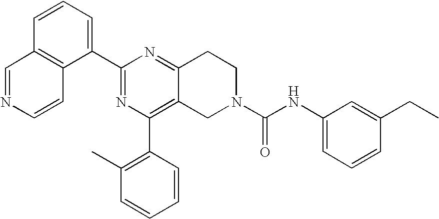 | 500.4 |
| 45 | 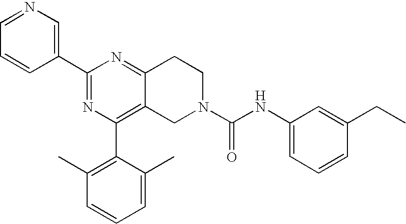 | 464.1 |
| 46 | 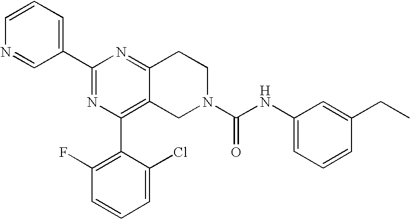 | 488.1 |
| 47 | 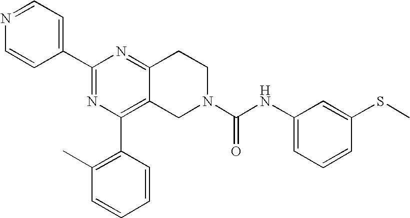 | 468.0 |
| 48 | 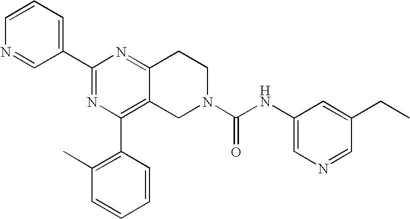 | 451.2 |
| 49 | 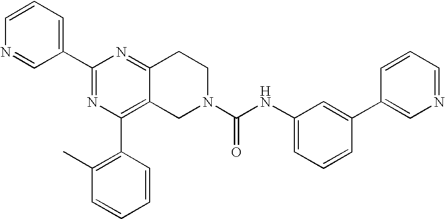 | 499.0 |
| 50 | 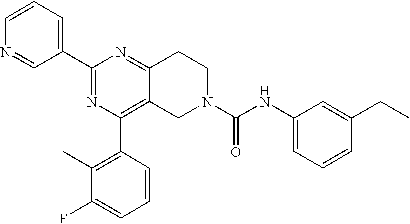 | 468.1 |
| 51 | 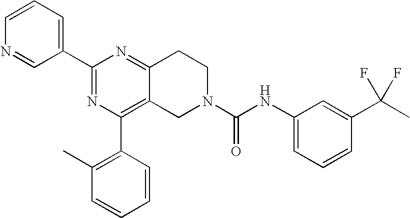 | 485.8 |
| 52 | 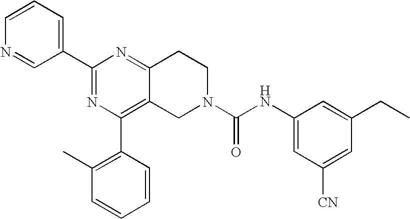 | 475.0 |
| 53 | 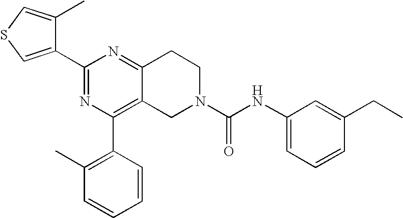 | 469.1 |
| 54 | 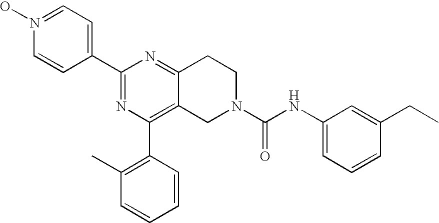 | 466.3 |
| 55 | 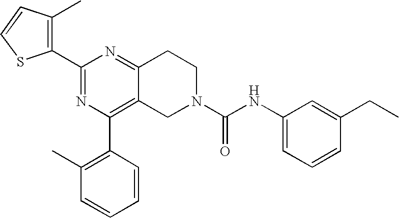 | 469.0 |
| 56 | 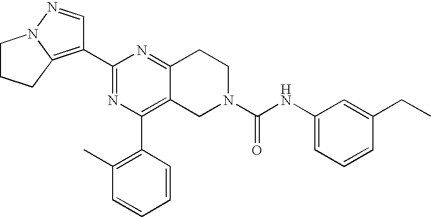 | 479.2 |
| 57 | 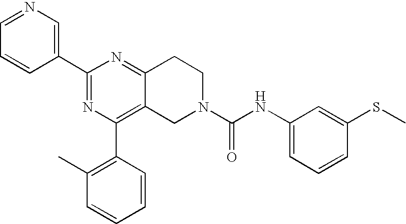 | 468.0 |
| 58 | 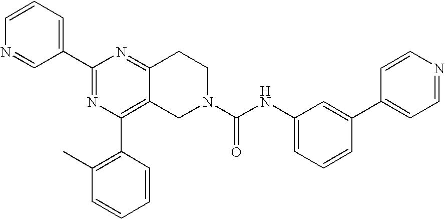 | 499.2 |
| 59 | 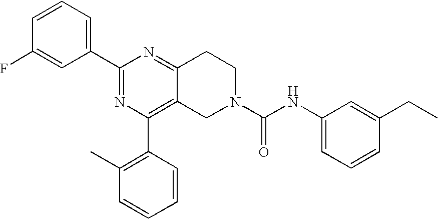 | 467.3 |
| 60 | 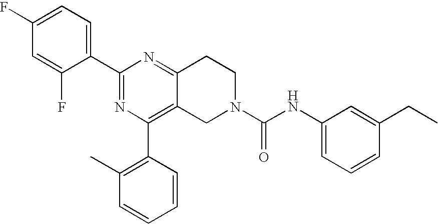 | 485.3 |
| 61 | 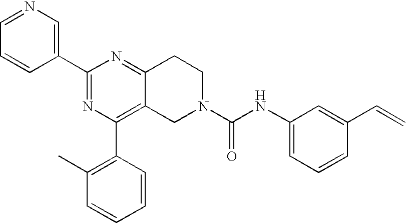 | 448.2 |
| 62 | 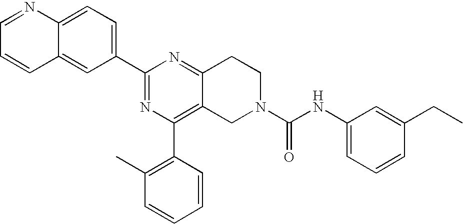 | 500.1 |
| 63 | 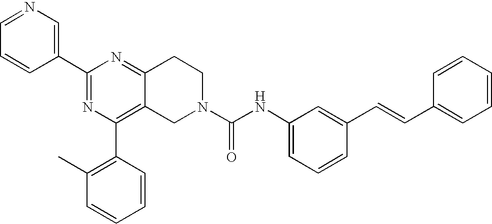 | 523.9 |
| 64 | 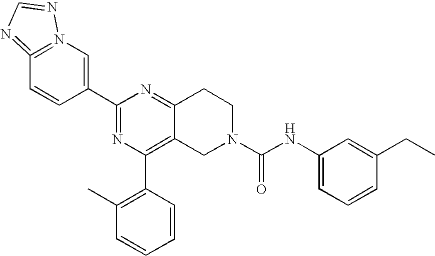 | 490.5 |
| 65 | 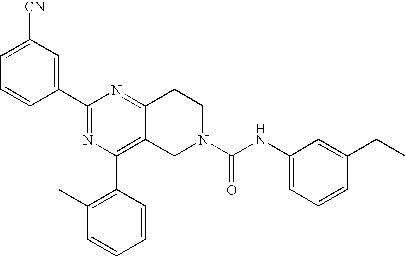 | 473.9 |
| 66 | 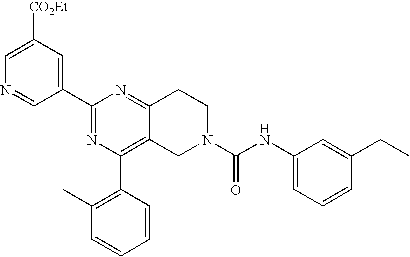 | 522.2 |
| 67 | 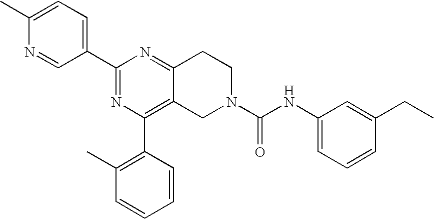 | 464.3 |
| 68 | 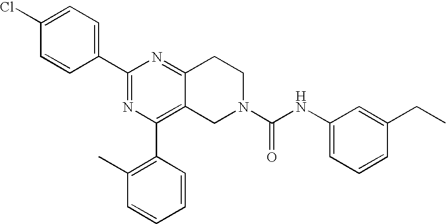 | 483.0 |
| 69 | 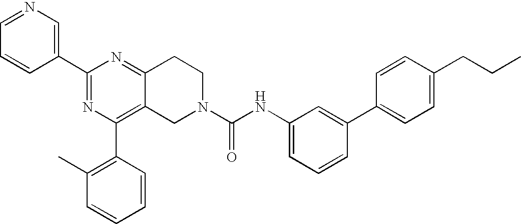 | 539.9 |
| 70 | 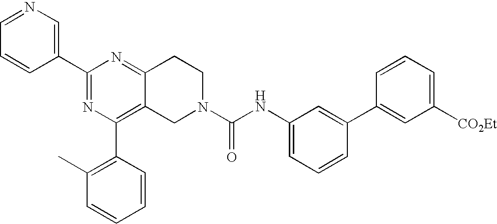 | 570.3 |
| 71 | 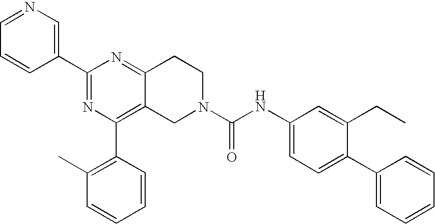 | 526.0 |
| 72 | 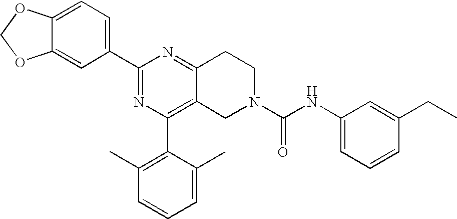 | 507.7 |
| 73 | 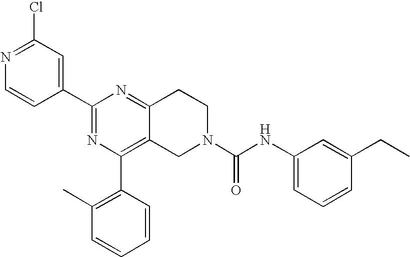 | 484.1 |
| 74 | 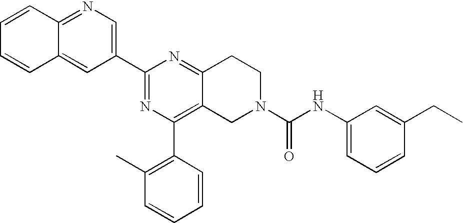 | 500.1 |
| 75 | 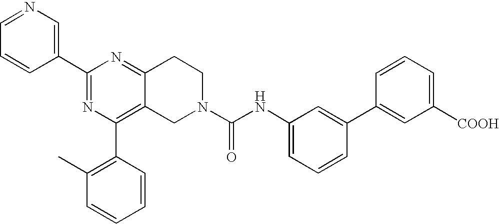 | 542.1 |
| 76 | 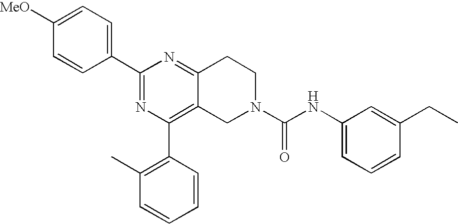 | 479.0 |
| 77 | 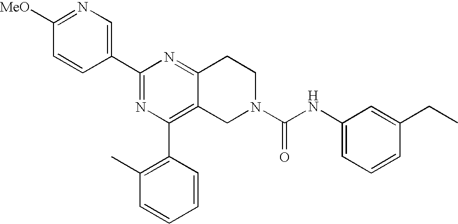 | 480.2 |
| 78 | 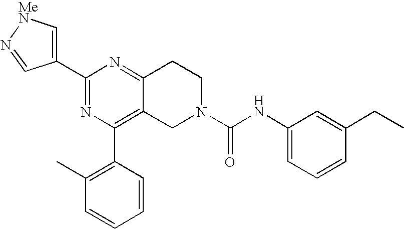 | 453.2 |
| 79 | 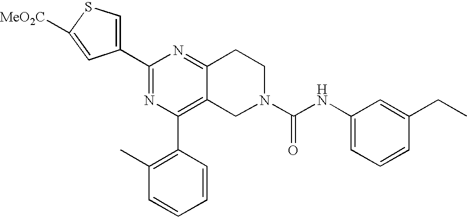 | 513.3 |
| 80 | 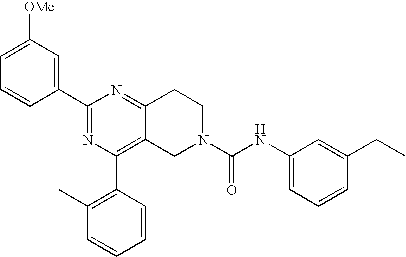 | 479.0 |
| 81 | 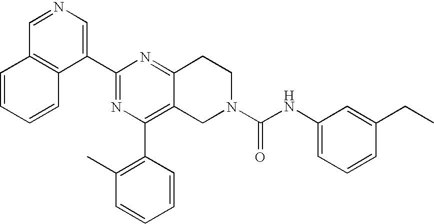 | 500.3 |
| 82 | 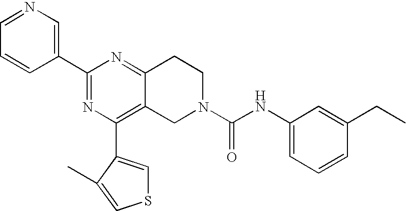 | 456.1 |
| 83 | 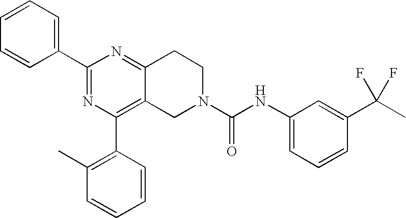 | 485.2 |
| 84 | 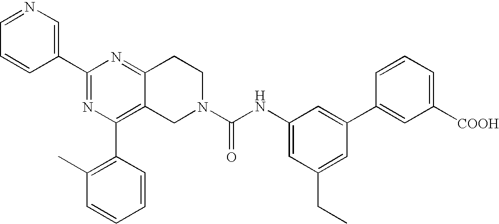 | 570.2 |
| 85 | 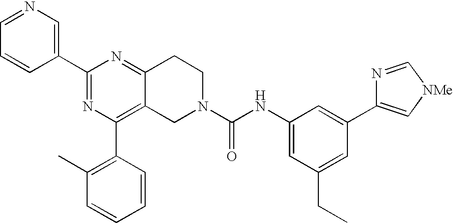 | 530.3 |
| 86 | 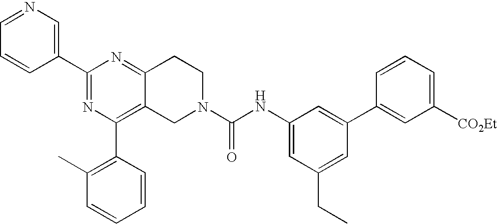 | 598.3 |
| 87 | 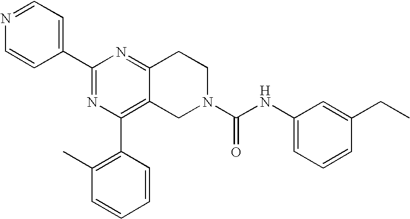 | 450.1 |
| 88 | 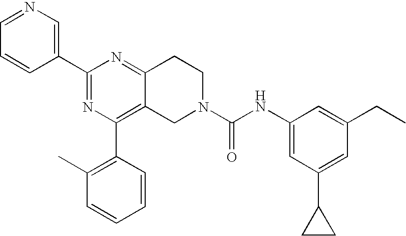 | 490.3 |
| 89 | 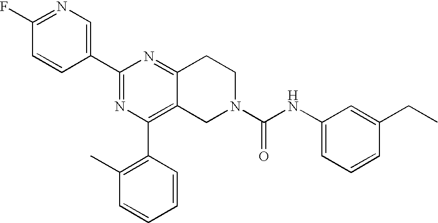 | 468.1 |
| 90 | 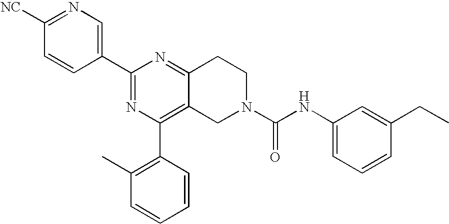 | 475.1 |
| 91 | 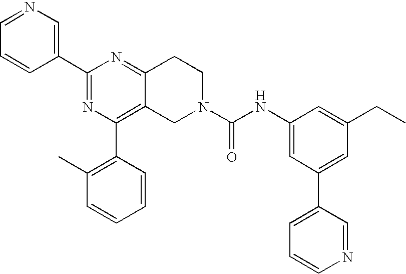 | 527.2 |
| 92 | 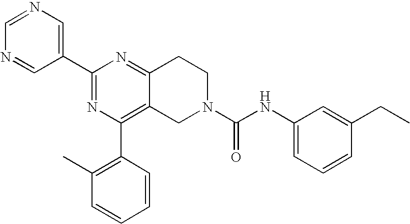 | 451.2 |
| 93 | 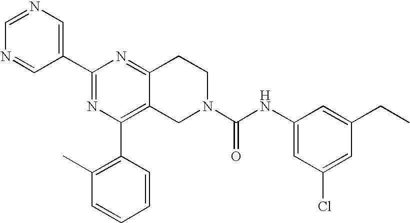 | 483.2 (M − H) |
| 94 | 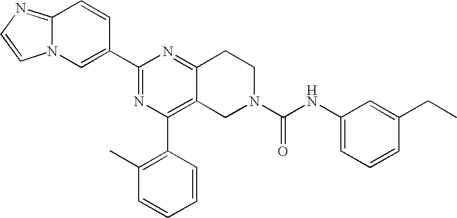 | 489.1 |
| 95 | 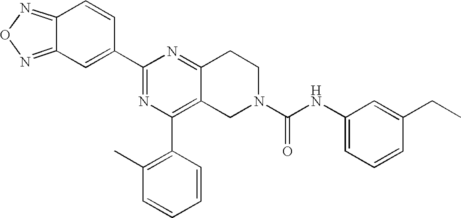 | 491.1 |
| 96 | 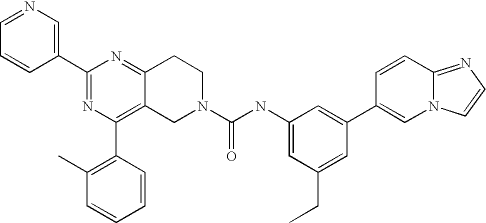 | 566.3 |
| 97 | 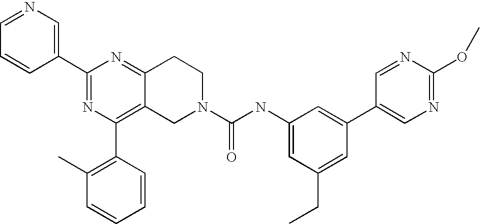 | 558.1 |
| 98 | 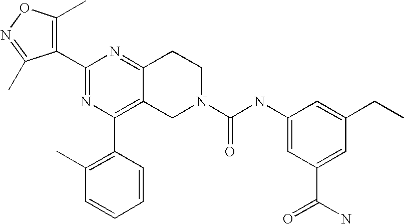 | 511.1 |
| 99 | 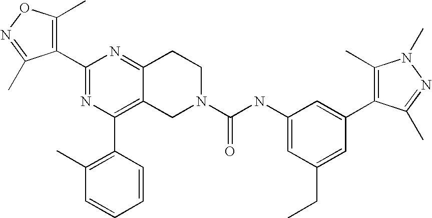 | 576.1 |
| 100 | 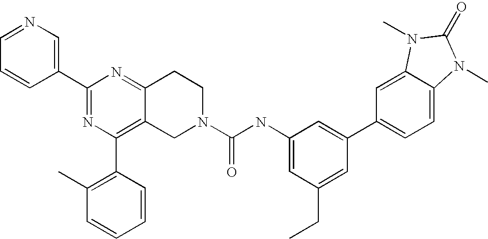 | 610.1 |
| 101 | 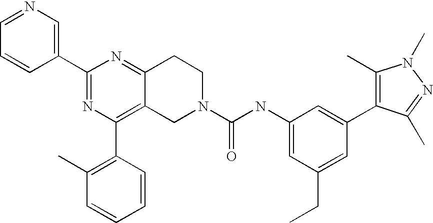 | 558.1 |
| 102 | 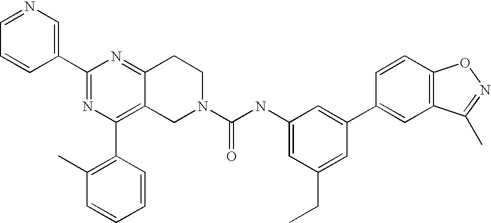 | 581.2 |
| 103 | 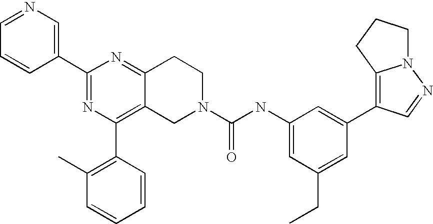 | 556.3 |
| 104 | 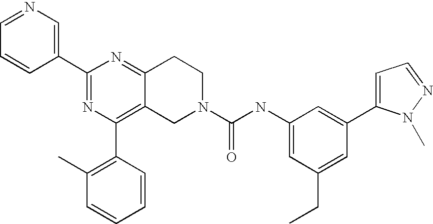 | 529.7 |
| 105 | 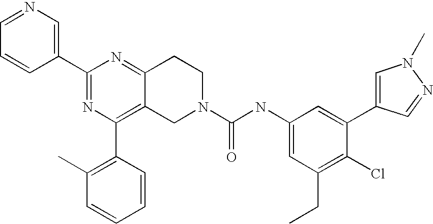 | 564.2 |
| 106 | 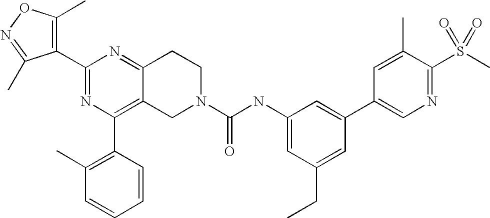 | 637.2 |
| 107 | 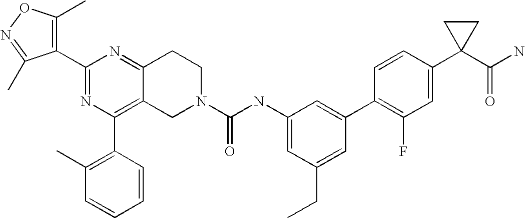 | 645.2 |
| 108 | 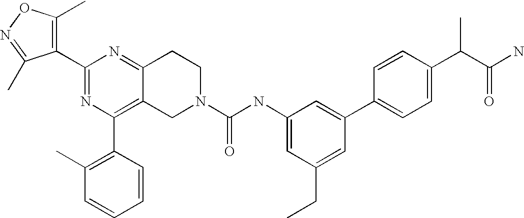 | 615.3 |
| 109 | 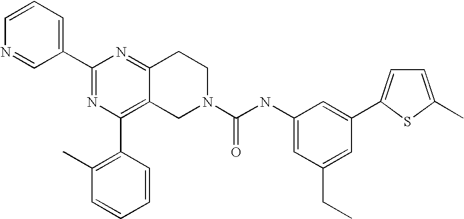 | 546.3 |
| 110 | 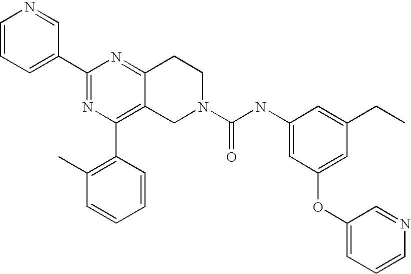 | 543.5 |
| 111 | 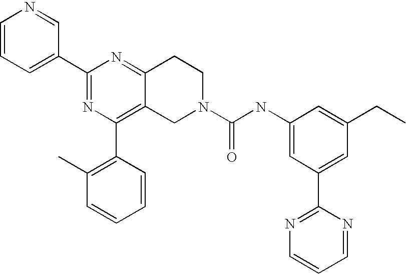 | 527.8 |
| 112 | 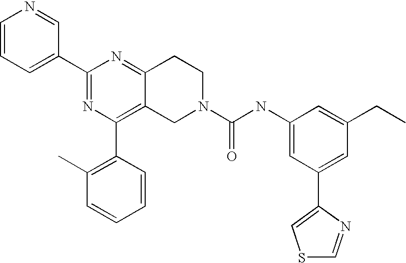 | 533.2 |
| 113 | 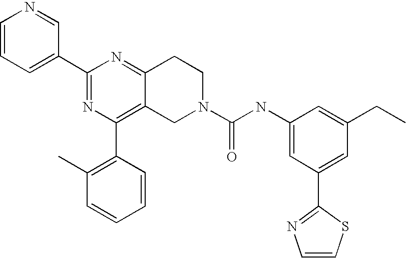 | 533.2 |
| 114 | 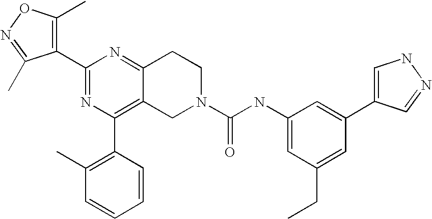 | 534.2 |
| 115 | 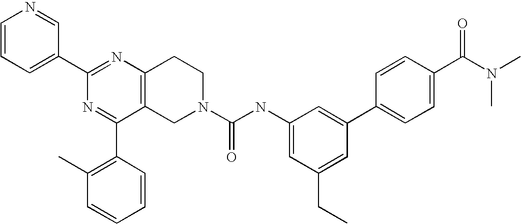 | 597.3 |
| 116 | 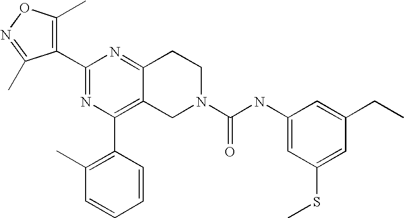 | 514.2 |
| 117 | 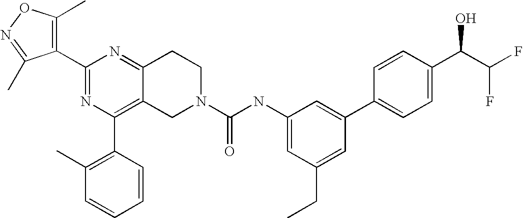 | 624.3 |
| 118 | 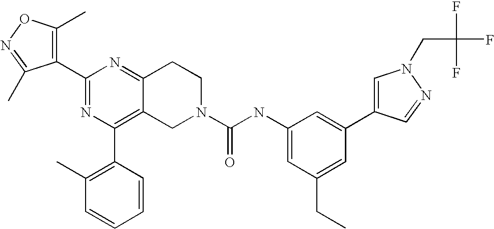 | 616.4 |
| 119 | 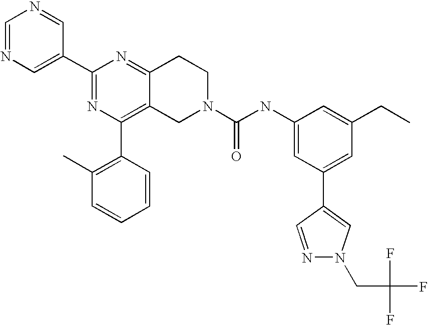 | 599.5 |
| 120 | 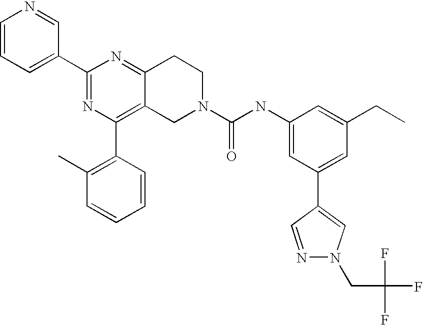 | 598.3 |
| 121 | 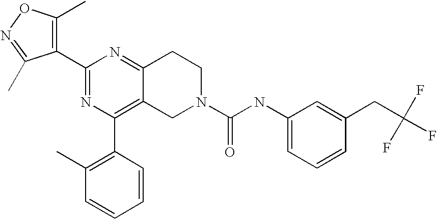 | 522.2 |
| 122 | 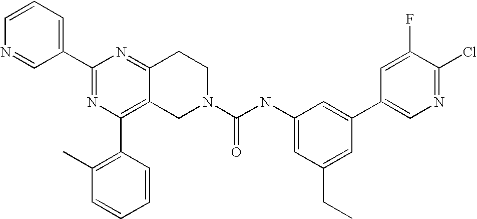 | 579.2 |
| 123 | 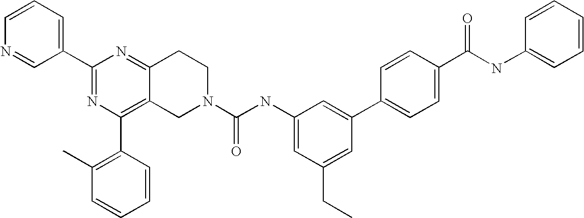 | 645.3 |
| 124 | 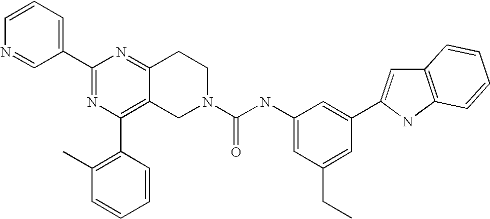 | 565.2 |
| 125 | 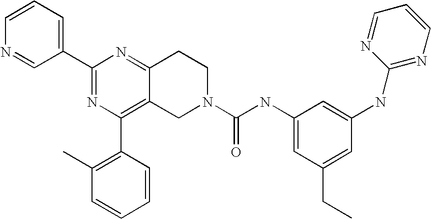 | 543.2 |
| 126 | 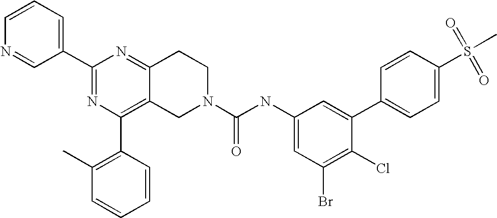 | 688.2/690.2 |
| 127 | 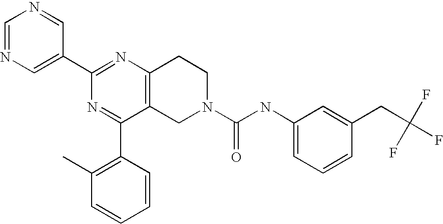 | 504.8 |
| 128 | 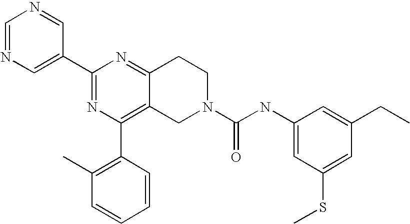 | 497.0 |
| 129 | 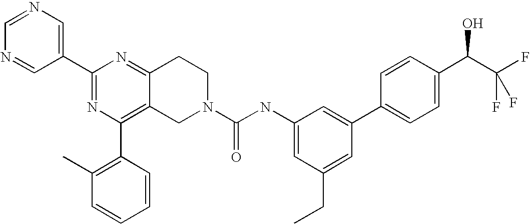 | 625.2 |
| 130 | 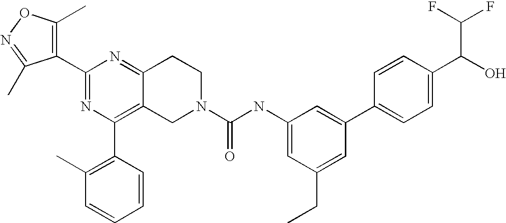 | 624.2 |
| 131 | 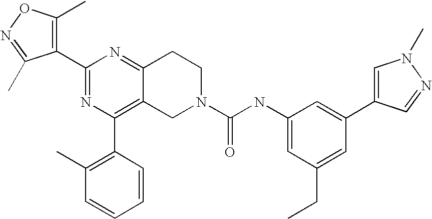 | 548.2 |
| 132 | 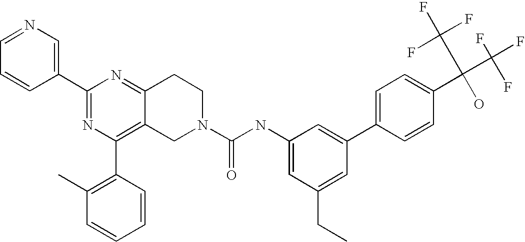 | 692.3 |
| 133 | 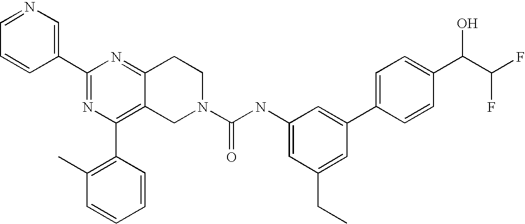 | 606.2 |
| 134 | 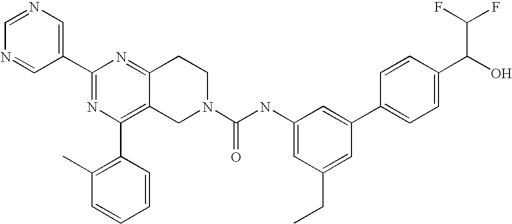 | 607.2 |
| 135 | 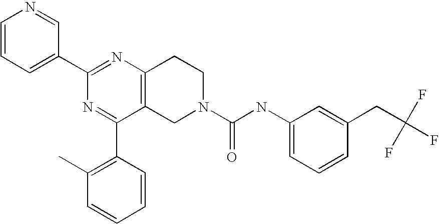 | 504.1 |
| 136 | 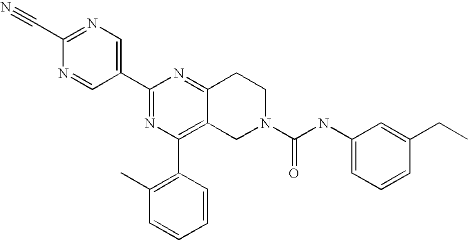 | 476.2 |
| 137 | 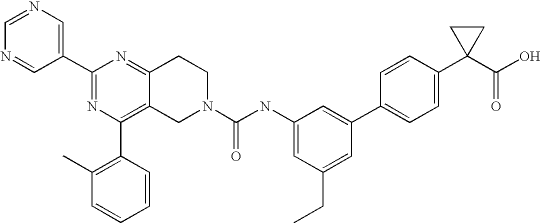 | 611.3 |
| 138 | 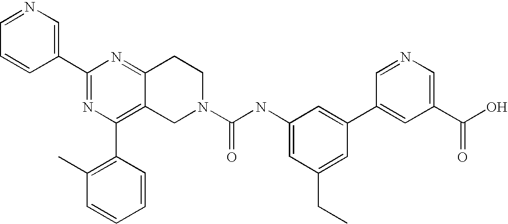 | 571.1 |
| 139 | 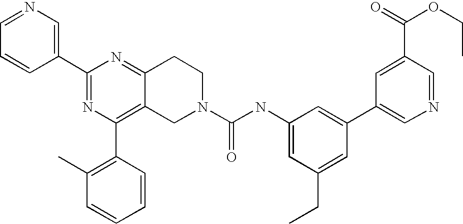 | 599.4 |
| 140 | 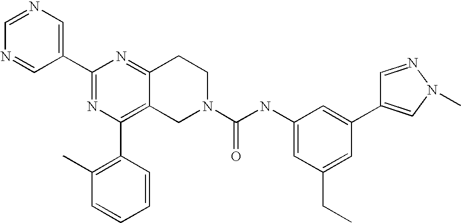 | 531.2 |
| 141 | 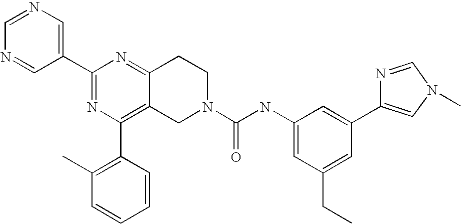 | 531.2 |
| 142 | 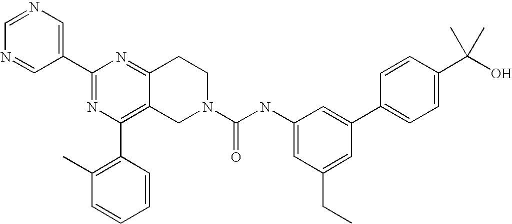 | 585.4 |
| 143 | 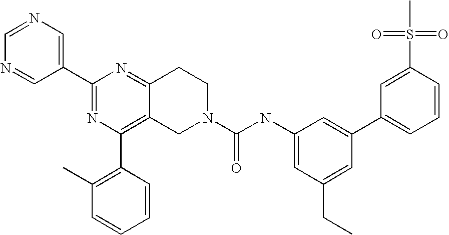 | 605.1 |
| 144 | 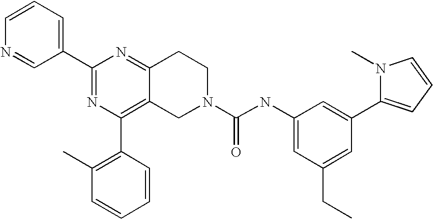 | 529.3 |
| 145 | 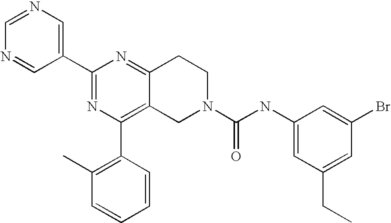 | 529.1/531.1 |
| 146 | 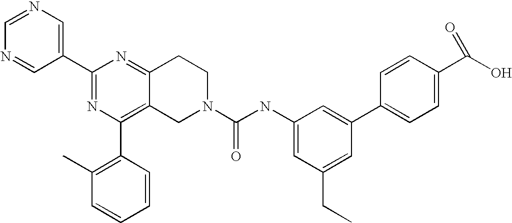 | 571.0 |
| 147 | 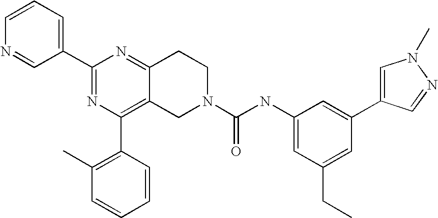 | 530.2 |
| 148 | 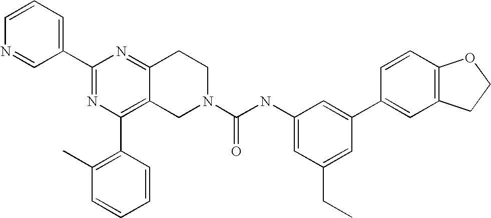 | 568.3 |
| 149 | 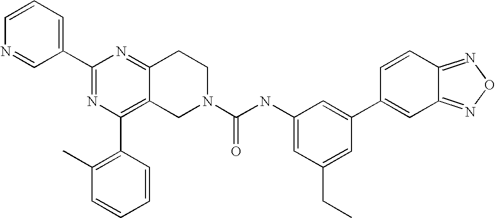 | 568.2 |
| 150 | 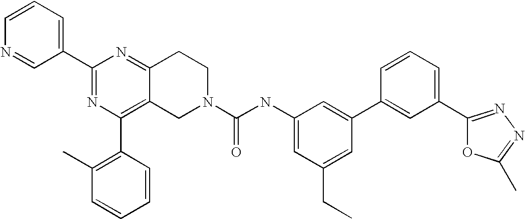 | 608.4 |
| 151 | 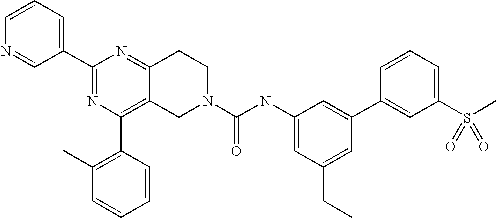 | 604.2 |
| 152 | 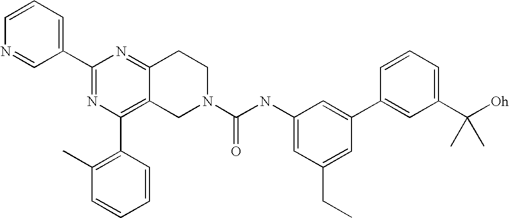 | 584.4 |
| 153 | 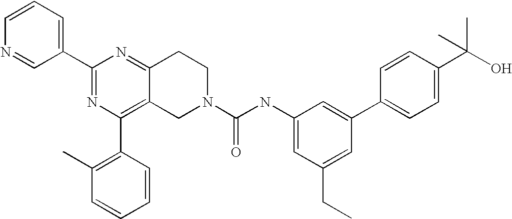 | 584.4 |

### Examples of Pharmaceutical Compositions

As a specific embodiment of an oral composition of a compound of the present invention, 50 mg of the compound of any of the Examples is formulated with sufficient finely divided lactose to provide a total amount of 580 to 590 mg to fill a size 0 hard gelatin capsule.

As a second specific embodiment of an oral composition of a compound of the present invention, 100 mg of the compound of any of the Examples, microcrystalline cellulose (124 mg), croscarmellose sodium (8 mg), and anhydrous unmilled dibasic calcium phosphate (124 mg) are thoroughly mixed in a blender; magnesium stearate (4 mg) and sodium stearyl fumarate (12 mg) are then added to the blender, mixed, and the mix transferred to a rotary tablet press for direct compression. The resulting tablets are optionally film-coated with Opadry® II for taste masking.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

1. A compound of structural formula I: 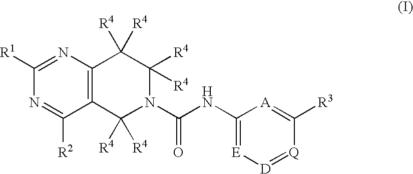
or a pharmaceutically acceptable salt thereof, wherein
A, Q, D, and E are each independently N or CR⁸, with the proviso that at least two of A, Q, D, and E represent CR⁸;
R¹ is aryl or heteroaryl wherein aryl and heteroaryl are optionally substituted with one to three substituents independently selected from R^{a}:
R^{a} is selected from the group consisting of:
cyano,
halogen,
C₁₋₆ alkyl, optionally substituted with one hydroxy and one to six fluorines,
C₂₋₆ alkenyl,
C₂₋₆ alkynyl,
C₁₋₆ alkoxy, optionally substituted with one to five fluorines,
C₁₋₆ alkylthio, optionally substituted with one to five fluorines,
C₁₋₆ alkylsulfonyl, optionally substituted with one to five fluorines,
(CH₂)ₙC₃₋₆ cycloalkyl, wherein cycloalkyl is optionally substituted with one to three substituents independently selected from halogen, hydroxy, cyano, nitro, CO₂H, C₁₋₆ alkyloxycarbonyl, C₁₋₆ alkyl, and C₁₋₆ alkoxy, wherein alkyl and alkoxy are optionally substituted with one to five fluorines,
(CH₂)ₙOR⁵,
(CH₂)ₙN(R⁵)₂,
(CH₂)ₙC≡N,
(CH₂)ₙCO₂R⁵,
(CH₂)ₙNR¹⁰SO₂R⁹,
(CH₂)ₙSO₂N(R⁵)₂,
(CH₂)ₙS(O)ᵣR⁵,
(CH₂)ₙNR¹⁰C(O)N(R⁵)₂,
(CH₂)ₙC(O)N(R⁵)₂,
(CH₂)ₙNR¹⁰C(O)R⁵,
(CH₂)ₙNR¹⁰CO₂R⁹,
(CH₂)ₙC(O)R⁵,
aryl, and
heteroaryl;
wherein aryl and heteroaryl are optionally substituted with one to three substituents independently selected from the group consisting of halogen, C₁₋₄ alkyl, —CO₂C₁₋₄ alkyl, and CF₃ and wherein any individual methylene (CH₂) carbon atom in (CH₂)ₙ is optionally substituted with one to two substituents independently selected from fluorine, hydroxy, C₁₋₄ alkyl, and C₁₋₄ alkoxy, wherein alkyl and alkoxy are optionally substituted with one to five fluorines; or two substituents when on the same methylene (CH₂) group are taken together with the carbon atom to which they are attached to form a cyclopropyl group;
R² is 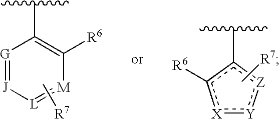
wherein R⁶ is selected from the group consisting of:
C₁₋₆ alkyl, optionally substituted with hydroxy, C₁₋₃ alkoxy, or one to five fluorines;
C₂₋₆ alkenyl,
C₂₋₆ alkynyl,
(CH₂)ₙ—C₃₋₆ cycloalkyl, wherein cycloalkyl is optionally substituted with one to three substituents independently selected from halogen, hydroxy, cyano, nitro, CO₂H, C₁₋₆ alkyloxycarbonyl, C₁₋₆ alkyl, and C₁₋₆ alkoxy, wherein alkyl and alkoxy are optionally substituted with one to five fluorines,
cyano,
halogen,
hydroxy,
C₁₋₄ alkoxy, optionally substituted with one to five fluorines, and
C₁₋₄ alkylthio, optionally substituted with one to five fluorines;
wherein any individual methylene (CH₂) carbon atom in (CH₂)ₙ is optionally substituted with one to two substituents independently selected from fluorine, hydroxy, C₁₋₄ alkyl, and C₁₋₄ alkoxy, wherein alkyl and alkoxy are optionally substituted with one to five fluorines; or two substituents when on the same methylene (CH₂) group are taken together with the carbon atom to which they are attached to form a cyclopropyl group;
G, J, L and M are each independently N or CR⁷, with the proviso that at least two of G, J, L and M represent CR⁷;
X, Y, and Z are each independently O, S, or N, with the proviso that the combination of X, Y, and Z cannot represent more than one O or S;
each R⁷ is independently selected from the group consisting of hydrogen, halogen, and C₁₋₄ alkyl optionally substituted with one to five fluorines;
R³ is selected from the group consisting of:
cyano,
halogen,
C₁₋₆ alkyl, optionally substituted with one to five fluorines,
C₂₋₆ alkenyl,
C₂₋₆ alkynyl,
C₁₋₆ alkoxy, optionally substituted with one to five fluorines,
C₁₋₆ alkylthio, optionally substituted with one to five fluorines,
C₁₋₆ alkylsulfonyl, optionally substituted with one to five fluorines,
(CH₂)ₙ—C₃₋₆ cycloalkyl, wherein cycloalkyl is optionally substituted with one to three substituents independently selected from halogen, hydroxy, cyano, nitro, CO₂H, C₁₋₆ alkyloxycarbonyl, C₁₋₆ alkyl, and C₁₋₆ alkoxy, wherein alkyl and alkoxy are optionally substituted with one to five fluorines,
(CH₂)ₙOR⁵,
(CH₂)ₙN(R⁵)₂,
(CH₂)ₙC≡N,
(CH₂)ₙCO₂R⁵,
(CH₂)ₙNR¹⁰SO₂R⁹,
(CH₂)ₙSO₂N(R⁵)₂,
(CH₂)ₙS(O)ᵣR⁵,
(CH₂)ₙNR¹⁰C(O)N(R⁵)₂,
(CH₂)ₙC(O)N(R⁵)₂,
(CH₂)ₙNR¹⁰C(O)R⁵,
(CH₂)ₙNR¹⁰CO₂R⁹,
(CH₂)ₙC(O)R⁵, CH═CH-aryl,
(CH₂)ₚ—W—(CH₂)_{q}-aryl, and
(CH₂)ₚ—W—(CH₂)_{q}-heteroaryl;
wherein W is a bond, O, S(O)ᵣ, or NR¹⁰; aryl and heteroaryl are optionally substituted with one to three R^{a} substituents; and any individual methylene (CH₂) carbon atom in (CH₂)ₙ, (CH₂)ₚ, or (CH₂)_{q} is optionally substituted with one to two substituents independently selected from fluorine, hydroxy, C₁₋₄ alkyl, and C₁₋₄ alkoxy, wherein alkyl and alkoxy are optionally substituted with one to five fluorines; or two substituents when on the same methylene (CH₂) group are taken together with the carbon atom to which they are attached to form a cyclopropyl group;
each R⁸ is selected from the group consisting of:
hydrogen,
cyano,
halogen,
C₁₋₆ alkyl, optionally substituted with one to five fluorines,
C₂₋₆ alkenyl,
C₂₋₆ alkynyl,
C₁₋₆ alkoxy, optionally substituted with one to five fluorines,
C₁₋₆ alkylthio, optionally substituted with one to five fluorines,
C₁₋₆ alkylsulfonyl, optionally substituted with one to five fluorines,
CH₂)ₙ—C₃₋₆ cycloalkyl, wherein cycloalkyl is optionally substituted with one to three substituents independently selected from halogen, hydroxy, cyano, nitro, CO₂H, C₁₋₆ alkyloxycarbonyl, C₁₋₆ alkyl, and C₁₋₆ alkoxy, wherein alkyl and alkoxy are optionally substituted with one to five fluorines,
(CH₂)ₙOR⁵,
(CH₂)ₙN(R⁵)₂,
(CH₂)ₙC≡N,
(CH₂)ₙCO₂R⁵,
(CH₂)ₙNR¹⁰SO₂R⁹,
(CH₂)ₙSO₂N(R⁵)₂,
(CH₂)ₙS(O)ᵣR⁵,
(CH₂)ₙNR¹⁰C(O)N(R⁵)₂,
(CH₂)ₙC(O)N(R⁵)₂,
(CH₂)ₙNR¹⁰C(O)R⁵,
(CH₂)ₙNR¹⁰CO₂R⁹,
(CH₂)ₙC(O)R⁵,
(CH₂)ₚ—W—(CH₂)_{q}-aryl, and
(CH₂)ₚ—W—(CH₂)_{q}-heteroaryl;
wherein W is a bond, O, S(O)ᵣ, or NR¹⁰; aryl and heteroaryl are optionally substituted with one to three R^{a} substituents; and any individual methylene (CH₂) carbon atom in (CH₂)ₙ, (CH₂)ₚ, or (CH₂)_{q} is optionally substituted with one to two substituents independently selected from fluorine, hydroxy, C₁₋₄ alkyl, and C₁₋₄ alkoxy, wherein alkyl and alkoxy are optionally substituted with one to five fluorines; or two substituents when on the same methylene (CH₂) group are taken together with the carbon atom to which they are attached to form a cyclopropyl group;
each R⁴ is independently hydrogen, fluorine, or C₁₋₃ alkyl; or two R⁴ groups together with the carbon atom to which they are attached can form a 3- to 6-membered carbocyclic ring system;
each R⁵ is independently selected from the group consisting of
hydrogen,
C₁₋₆ alkyl, optionally substituted with one to five fluorines,
(CH₂)ₘ-aryl,
(CH₂)ₘ-heteroaryl, and
(CH₂)ₘC₃₋₆ cycloalkyl;
wherein any individual methylene (CH₂) carbon atom in (CH₂)ₘ is optionally substituted with one to two substituents independently selected from fluorine, hydroxy, C₁₋₄ alkyl, and C₁₋₄ alkoxy, wherein alkyl and alkoxy are optionally substituted with one to five fluorines; or two substituents when on the same methylene (CH₂) group are taken together with the carbon atom to which they are attached to form a cyclopropyl group; and wherein alkyl, aryl, heteroaryl, and cycloalkyl are optionally substituted with one to three groups independently selected from halogen, C₁₋₄ alkyl, and C₁₋₄ alkoxy; or two R⁵ groups substituents together with the nitrogen atom to which they are attached form a heterocyclic ring selected from azetidine, pyrrolidine, piperidine, piperazine, and morpholine wherein said heterocyclic ring is unsubstituted or substituted with one to three substituents independently selected from halogen, hydroxy, C₁₋₆ alkyl, and C₁₋₆ alkoxy, wherein alkyl and alkoxy are optionally substituted with one to five fluorines;
each R⁹ is independently C₁₋₆ alkyl, wherein alkyl is optionally substituted with one to five substituents independently selected from fluorine and hydroxy;
R¹⁰ is hydrogen or R⁹;
each n is independently an integer from 0 to 3;
each m is independently an integer from 0 to 2;
each p is an integer from 0 to 2;
each q is an integer from 0 to 2; and
each r is an integer from 0 to 2.

2. The compound of claim 1 wherein R¹ is a phenyl group, a 5- or 6-membered monocyclic heteroaryl group, or a 9- or 10-membered bicyclic heteroaryl group containing one to three heteroatoms selected from O, S, and N, wherein the phenyl or heteroaryl group is optionally substituted with one to two substituents independently selected from R^{a}.

3. The compound of claim 2 wherein R¹ is a heteroaryl group selected from the group consisting of pyridinyl, N-oxo-pyridinyl, pyrimidinyl, isoxazolyl, thienyl, 1,3-benzodioxolyl, quinolyl, and pyrazolyl, each of which is optionally substituted with one to two substituents independently selected from R^{a}.

4. The compound of claim 3 wherein R¹ is pyridinyl or pyrimidinyl, each of which is optionally substituted with one to two substituents independently selected from R^{a}.

5. The compound of claim 2 wherein R¹ is phenyl optionally substituted with one to two substituents independently selected from R^{a}.

6. The compound of claim 1 wherein R² is 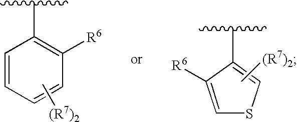
wherein R⁶ is selected from the group consisting of C₁₋₃ alkyl, chlorine, and bromine.

7. The compound of claim 6 wherein R² is

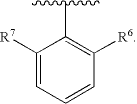

8. The compound of claim 7 wherein R⁶ is methyl or chlorine and
R⁷ is hydrogen, methyl, chlorine, or fluorine.

9. The compound of claim 1 wherein A and E are CH; D is N or CR⁸; and Q is CR⁸.

10. The compound of claim 9 wherein R³ is selected from the group consisting of:
—CH₂—C₁₋₅ alkyl, wherein —CH₂— is optionally substituted with one to two fluorines and alkyl is optionally substituted with one to five fluorines,
—C₃₋₆ cycloalkyl,
—C₁₋₄ alkenyl,
—C₁₋₄ alkoxy, optionally substituted with one to five fluorines,
C₁₋₄ alkylthio, optionally substituted with one to five fluorines,
—CH₂-aryl,
—CH₂CH₂-aryl,
—W-aryl, and
—W-heteroaryl;
wherein W is a bond, O, or S; and aryl and heteroaryl are optionally substituted with one to three R^{a} substituents.

11. The compound of claim 10 wherein R³ is ethyl, optionally substituted with one to five fluorines, and R⁸ is selected from the group consisting of:
hydrogen,
halogen,
cyano,
C₁₋₃ alkyl, optionally substituted with one to five fluorines,
C₃₋₅ cycloalkyl,
—W-phenyl, and
—W-heteroaryl;
wherein W is a bond, O, or S; and aryl and heteroaryl are optionally substituted with one to three R^{a} substituents.

12. The compound of claim 10 wherein R³ is phenyl, optionally substituted with one to three R^{a} substituents.

13. The compound of claim 1 wherein A, E, and Q are CH; D is N or CR⁸; and R³ is selected from the group consisting of:
—CH₂—C₁₋₅ alkyl, wherein —CH₂— is optionally substituted with one to two fluorines and alkyl is optionally substituted with one to five fluorines,
—C₃₋₆ cycloalkyl,
—C₁₋₄ alkenyl,
—C₁₋₄ alkoxy, optionally substituted with one to five fluorines,
—C₁₋₄ alkylthio, optionally substituted with one to five fluorines,
—CH₂-aryl,
—CH₂CH₂-aryl,
—W-aryl, and
—W-heteroaryl;
wherein W is a bond, O, or S; and aryl and heteroaryl are optionally substituted with one to three R^{a} substituents.

14. The compound of claim 13 wherein R³ is ethyl, optionally substituted with one to five fluorines, and R⁸ is selected from the group consisting of:
hydrogen,
halogen,
cyano,
C₁₋₃ alkyl, optionally substituted with one to five fluorines,
C₃₋₅ cycloalkyl,
—W-phenyl, and
—W-heteroaryl;
wherein W is a bond, O, or S; and aryl and heteroaryl are optionally substituted with one to three R^{a} substituents.

15. The compound of claim 1 wherein A, E, and D are CH; Q is CR⁸; and R³ is selected from the group consisting of:
—CH₂—C₁₋₅ alkyl, wherein —CH₂— is optionally substituted with one to two fluorines and alkyl is optionally substituted with one to five fluorines,
—C₃₋₆ cycloalkyl,
—C₁₋₄ alkenyl,
—C₁₋₄ alkoxy, optionally substituted with one to five fluorines,
—C₁₋₄ alkylthio, optionally substituted with one to five fluorines,
—CH₂-aryl,
—CH₂CH₂-aryl,
—W-aryl, and
—W-heteroaryl;
wherein W is a bond, O, or S; and aryl and heteroaryl are optionally substituted with one to three R^{a} substituents.

16. The compound of claim 15 wherein R³ is ethyl, optionally substituted with one to five fluorines, and R⁸ is selected from the group consisting of:
hydrogen,
halogen,
cyano,
C₁₋₃ alkyl, optionally substituted with one to five fluorines,
C₃₋₅ cycloalkyl,
—W-phenyl, and
—W-heteroaryl;
wherein W is a bond, O, or S; and aryl and heteroaryl are optionally substituted with one to three R^{a} substituents.

17. The compound of claim 1 which is selected from the group consisting of: 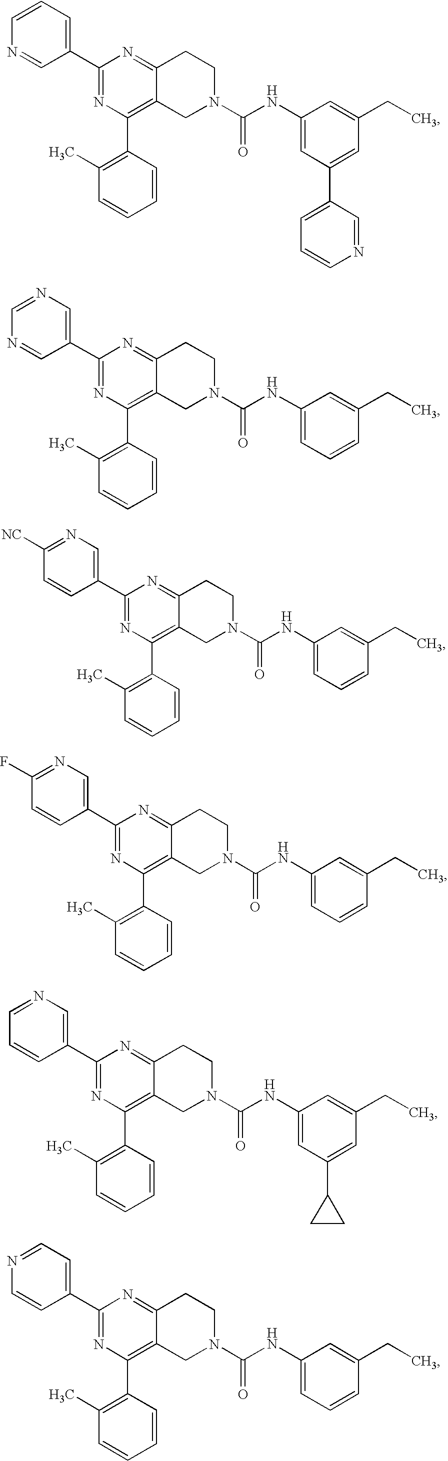 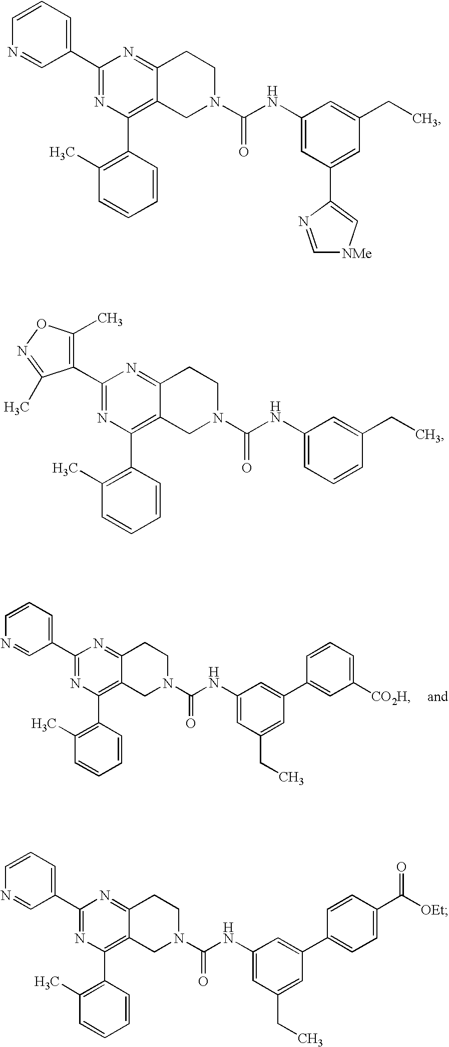
or a pharmaceutically acceptable salt thereof.

18. A pharmaceutical composition comprising a compound in accordance with claim 1 in combination with a pharmaceutically acceptable carrier.

19-20. (canceled)

21. A method for treating non-insulin dependent (Type 2) diabetes, insulin resistance, hyperglycemia, a lipid disorder, and obesity in a mammal in need thereof which comprises the administration to the mammal of a therapeutically effective amount of a compound of claim 1.
